SEQUENCE LISTING (55 sequences)

SEQ ID NO: 1 - protein, 10 aa, Mus musculus
GYTFTDHTIH

SEQ ID NO: 2 - protein, 17 aa, Mus musculus
YNYPRDGSTKYNEKFKG

SEQ ID NO: 3 - protein, 11 aa, Mus musculus
GFITTVVPSAY

SEQ ID NO: 4 - protein, 11 aa, Mus musculus
RASKSISKYLA

SEQ ID NO: 5 - protein, 7 aa, Mus musculus
SGSTLQS

SEQ ID NO: 6 - protein, 9 aa, Mus musculus
QQHNEYPWT

SEQ ID NO: 7 - protein, 120 aa, Mus musculus
QVQLQQSDAELVKPGASVKISCKVSGYTFTDHTIHWMKQRPEQGLEWIGYNYPRDGSTKYNEKFKGKATLTADKSSSTAYMQLNSLTSEDSAVYFCAKGFITTVVPSAYWGQGTLVTVSA

SEQ ID NO: 8 - protein, 107 aa, Mus musculus
DVQITQSPSYLAASPGETITINCRASKSISKYLAWYQEKPGKTNKLLIYSGSTLQSGIPSRFSGSGSGTDFTLTISSLEPEDFAMYYCQQHNEYPWTFGGGTKLEIK

SEQ ID NO: 9 - protein, 10 aa, Artificial Sequence
  6C8 HCDR1
GYAFSSYWMN

SEQ ID NO: 10 - protein, 17 aa, Artificial Sequence
  6C8 HCDR2
QIYPGDGDTNYNGKFKG

SEQ ID NO: 11 - protein, 10 aa, Artificial Sequence
  6C8 HCDR3
RCYYGLYFDV

SEQ ID NO: 12 - protein, 11 aa, Artificial Sequence
  6C8 LCDR1
KASQDINRYLS

SEQ ID NO: 13 - protein, 7 aa, Artificial Sequence
  6C8 LCDR2
RANRLVD

SEQ ID NO: 14 - protein, 9 aa, Artificial Sequence
  6C8 LCDR3
LQYDEFPYT

SEQ ID NO: 15 - protein, 119 aa, Artificial Sequence
  6C8 VH
QVQLQQSGAELVKPGASVKISCKASGYAFSSYWMNWVKQRPGKGLEWIGQIYPGDGDTNYNGKFKGKATLTADKSSSTAYMQLSSLTSEDSAVYFCTRRCYYGLYFDVWGTGTTVTVSS

SEQ ID NO: 16 - protein, 108 aa, Artificial Sequence
  6C8 VL
DIKMTQSPSSRYASLGERVTITCKASQDINRYLSWFQQKPGKSPKTLIYRANRLVDGVPSSRFSGSGSGQDYSLTISSLEYEDMGIYYCLQYDEFPYTFGGGTKLEIK

SEQ ID NO: 17 - protein, 10 aa, Artificial Sequence
  7B10 HCDR1
GYTFTSYWMH

SEQ ID NO: 18 - protein, 16 aa, Artificial Sequence
  7B10 HCDR2
YINPSSGYTKYNQFKD

SEQ ID NO: 19 - protein, 8 aa, Artificial Sequence
  7B10 HCDR3
GGYYGFAY

SEQ ID NO: 20 - protein, 10 aa, Artificial Sequence
  7B10 LCDR1
SASSSVSYMY

SEQ ID NO: 21 - protein, 7 aa, Artificial Sequence
  7B10 LCDR2
LTSNLAS

SEQ ID NO: 22 - protein, 9 aa, Artificial Sequence
  7B10 LCDR3
QQWSSNPLT

SEQ ID NO: 23 - protein, 117 aa, Artificial Sequence
  7B10 VH
QVQLQQSGAELAKPGASVKLSCKASGYTFTSYWMHWVKQRPGQGLEWIGYINPSSGYTKYNQKFKDKATLTADKSSSTAYMQLSSLTYEDSAVYYCARGGYYGFAYWGQGTLVTVSA

SEQ ID NO: 24 - protein, 106 aa, Artificial Sequence
  7B10 VL
QIVLTQSPALMSASPGEKVTMTCSASSSVSYMYWYQQKPRSSPKPWIYLTSNLASGVPARFSGSGSGTSYSLTISSMEAEDAATYYCQQWSSNPLTFGAGTKLELK

SEQ ID NO: 25 - protein, 10 aa, Artificial Sequence
  9H10 HCDR1
GFSLTGYGVH

SEQ ID NO: 26 - protein, 16 aa, Artificial Sequence
  9H10 HCDR2
VIWSGGSTDYNAAFIS

SEQ ID NO: 27 - protein, 10 aa, Artificial Sequence
  9H10 HCDR3
NYGYDYAMDY

SEQ ID NO: 28 - protein, 11 aa, Artificial Sequence
  9H10 LCDR1
RASQSIGTSIH

SEQ ID NO: 29 - protein, 7 aa, Artificial Sequence
  9H10 LCDR2
YASESIS

SEQ ID NO: 30 - protein, 9 aa, Artificial Sequence
  9H10 LCDR3
QQSNSWPFT

SEQ ID NO: 31 - protein, 116 aa, Artificial Sequence
  9H10 VH
QVQLKQSGPGVQPSSLSITCTVSGFSLTSYGVHWVRQSPGKGLEWLGVIWSGGSTDYNAAFISRLSISKDNSKSQVFFKMNSLQADDTAIYYCARNYGYDYAMDYWGQGTSVTVSS

SEQ ID NO: 32 - protein, 107 aa, Artificial Sequence
  9H10 VL
DILLTQSPAILSVSPGERVSFSCRASQSIGTSIHWYQQRTNGSPRLLIKYASESISGIPSRFSGSGSGTDFTLSINSVESEDIADYYCQQSNSWPFTFGSGTKLEIK

SEQ ID NO: 33 - protein, 5 aa, Mus musculus
NYWMH

SEQ ID NO: 34 - protein, 17 aa, Mus musculus
AIYPGDSDTSYNQKFKG

SEQ ID NO: 35 - protein, 6 aa, Mus musculus
LYYGYD

SEQ ID NO: 36 - protein, 16 aa, Mus musculus
KSSQSLIDSDGKTFLN

SEQ ID NO: 37 - protein, 7 aa, Mus musculus
LVSKLDS

SEQ ID NO: 38 - protein, 9 aa, Mus musculus
WQGTHFPYT

SEQ ID NO: 39 - protein, 115 aa, Mus musculus
EVQLQQSGTVLARPGASVKMSCKASGYTFTNYWMHWVKQRPVQGLEWIGAIYPGDSDTSYNQKFKGKAKLTAVTSTSTAFMELSSLTNEDSAVYYCTGLYYGYDWGQGTTLTVSS

SEQ ID NO: 40 - protein, 112 aa, Mus musculus
DVLMTQTPLTLSVTIGQPASISCKSSQSLIDSDGKTFLNWLLQRPGQSPKRLIYLVSKLDSGVPDRFTGSGSGTDFTLKISRVEAEDLGLYYCWQGTHFPYTFGGGTKLEIK

SEQ ID NO: 41 - protein, 463 aa, Artificial Sequence
  humanized 2A2 (h2A2) Heavy chain
MDWTWRVFCLLAVAPGAHSQVQLVQSGAEVKKPGASVKVSCKASGYTFTNYWMHWVRQAPGQGLEWMGAIYPGDSDTSYNQKFKGRVTMTRDTSTSTVYMELSSLRSEDTAVYYCARLYYGYDWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 42 - protein, 239 aa, Artificial Sequence
  humanized 2A2 (h2A2) Light chain
MRLPAQLLGLLMLWVPGSSGDVVMTQSPLSLPVTLGQPASISCKSSQSLIDSDGKTFLNWFQQRPGQSPRRLIYLVSKLDSGVPDRFSGSGSGTDFTLKISRVEAEDVGVYYCWQGTHFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 10 aa, Artificial Sequence
  h6B5 HCDR1 (IH-MH)
GYTFTDHTMH

SEQ ID NO: 44 - protein, 17 aa, Artificial Sequence
  h6B5 HCDR2 (DG)
YNYPRDGSTKYNEKFQG

SEQ ID NO: 45 - protein, 17 aa, Artificial Sequence
  h6B5 HCDR2 (DG-EG)
YNYPREGSTKYNEKFQG

SEQ ID NO: 46 - protein, 17 aa, Artificial Sequence
  h6B5 HCDR2 (DG-DV)
YNYPRDVSTKYNEKFQG

SEQ ID NO: 47 - protein, 17 aa, Artificial Sequence
  h6B5 HCDR2 (NE-AE)
YNYPRDGSTKYAEKFQG

SEQ ID NO: 48 - protein, 120 aa, Artificial Sequence
  h6B5 VH-1
QVQLVQSGAEVKKPGASVKVSCKASGYTFTDHTMHWVRQAPGQGLEWMGYNYPRDGSTKYAEKFQGRVTMTADKSTSTVYMELSSLRSEDTAVYYCAKGFITTVVPSAYWGQGTLVTVSS

SEQ ID NO: 49 - protein, 120 aa, Artificial Sequence
  h6B5 VH-2
QVQLVQSGAEVKKPGASVKVSCKVSGYTFTDHTIHWMRQAPGQGLEWMGYNYPRDGSTKYNEKFQGRVTMTADKSTSTVYMELSSLRSEDTAVYYCAKGFITTVVPSAYWGQGTLVTVSS

SEQ ID NO: 50 - protein, 120 aa, Artificial Sequence
  h6B5 VH-3
QVQLVQSGAEVKKPGATVKISCKVSGYTFTDHTIHWMQQAPGKGLEWMGYNYPRDGSTKYNEKFQGRVTITADKSTSTAYMELSSLRSEDTAVYYCAKGFITTVVPSAYWGQGTLVTVSS

SEQ ID NO: 51 - protein, 120 aa, Artificial Sequence
  h6B5 VH-4
QVQLVQSGAEVKKPGASVKVSCKVSGYTFTDHTIHWMRQAPGQGLEWMGYNYPREGSTKYNEKFQGRVTMTADKSTSTVYMELSSLRSEDTAVYYCAKGFITTVVPSAYWGQGTLVTVSS

SEQ ID NO: 52 - protein, 120 aa, Artificial Sequence
  h6B5 VH-5
QVQLVQSGAEVKKPGASVKVSCKVSGYTFTDHTIHWMRQAPGQGLEWMGYNYPRDVSTKYNEKFQGRVTMTADKSTSTVYMELSSLRSEDTAVYYCAKGFITTVVPSAYWGQGTLVTVSS

SEQ ID NO: 53 - protein, 107 aa, Artificial Sequence
  h6B5 VL-1
DIQLTQSPSFLSASVGDRVTITCRASKSISKYLAWYQQKPGKAPKLLIYSGSTLQSGVPSRFSGSGSGTEFTLTISSLQPEDFATYYCQQHNEYPWTFGGGTKVEIK

SEQ ID NO: 54 - protein, 107 aa, Artificial Sequence
  h6B5 VL-2
DVQITQSPSFLSASVGDRVTITCRASKSISKYLAWYQQKPGKANKLLIYSGSTLQSGVPSRFSGSGSGTDFTLTISSLQPEDFATYYCQQHNEYPWTFGGGTKVEIK

SEQ ID NO: 55 - protein, 107 aa, Artificial Sequence
  h6B5 VL-3
DVQLTQSPSSVSASVGDRVTITCRASKSISKYLAWYQQKPGKAPKLLIYSGSTLQSGVPSRFSGSGSGTDFTLTISSLQPEDFATYYCQQHNEYPWTFGPGTKVEIK

(19) United States
(12) Patent Application Publication
(10) Pub. No.: US 2024/0158487 A1
(43) Pub. Date: May 16, 2024

(54) WOUND HEALING ENHANCEMENT WITH ANTI-CERAMIDE ANTIBODIES

(71) Applicants: MEMORIAL SLOAN-KETTERING CANCER CENTER, New York, NY (US); BOARD OF TRUSTEES OF MICHIGAN STATE UNIVERSITY, East Lansing, MI (US); MEMORIAL HOSPITAL FOR CANCER AND ALLIED DISEASES, New York, NY (US); SLOAN-KETTERING INSTITUTE FOR CANCER RESEARCH, New York, NY (US)

(72) Inventors: Richard Kolesnick, New York, NY (US); Julia Busik, East Lansing, MI (US)

(21) Appl. No.: 18/550,933

(22) PCT Filed: Mar. 15, 2022
(86) PCT No.: PCT/US2022/020385

§ 371 Date: Sep. 15, 2023

Related U.S. Application Data
(60) Provisional application No. 63/161,758, filed on Mar. 16, 2021.

(51) Int. Cl. C07K 16/18; A61K 39/00; A61P 17/02

(52) U.S. Cl. CPC C07K 16/18; A61P 17/02; A61K 2039/505; C07K 2317/24; C07K 2317/565; C07K 2317/622

(57) ABSTRACT

The present disclosure provides compositions and methods to improve or accelerate the healing of a wound. In various embodiments, the methods comprise the use of anti-ceramide antibodies and antibody fragments. In some embodiments, the wound is a chronic wound. In some embodiments, the wound is a diabetic wound.

### CROSS-REFERENCE TO RELATED APPLICATIONS

This application is a national stage application of International Application No. PCT/US2022/020385, filed Mar. 15, 2022, which claims the benefit of and priority to U.S. Provisional Application No. 63/161,758, filed on Mar. 16, 2021, the contents of each of which are herein incorporated by reference in their entirety.

### STATEMENT REGARDING SEQUENCE LISTING

The Sequence Listing associated with this application is provided in text format in lieu of a paper copy and is hereby incorporated by reference into the specification. A computer readable format copy of the Sequence Listing: filename: CERA_020_00US_SeqList_ST25.txt, date recorded: Mar. 16, 2021, file size about 31.7 kilobytes.

### FIELD

The present disclosure relates to anti-ceramide compositions and methods of use thereof for treating or preventing a wound, or enhancement of wound healing, for example diabetic wound healing.

### BACKGROUND

Diabetes affects 340 million people in the world, including 29.1 million individuals in the United States. A complication in diabetic patients is the inability of wounds to heal, which resulted in 73,000 lower-limb amputations in the United States in 2010.

In diabetic patients, high blood sugar triggers prolonged chronic inflammation, poor circulation, and neuropathy. The combination of these factors slows or even stops the wound healing process. Despite extensive research in this area, the underlying mechanism of impaired healing of diabetic wounds is multifactorial and remains poorly understood.

The standard treatment for diabetic wounds includes debridement of the wound, treatment of infection with antibiotics, and reducing or eliminating weight pressure from the lower extremities. However, there remains an unmet need for safe and effective treatments for accelerated wound healing, particularly for diabetic wounds.

### BRIEF DESCRIPTION OF DRAWINGS

FIG. 1A-FIG. 1B show representative photos of wound healing over time. Each row shows images of a particular lesion site in the indicated experimental group.

FIG. 2 shows the average rate of wound healing in each experimental group over time The Y axis indicates the size of the wound, with the starting size of each wound normalized as 1. The X axis indicates number of days. “D+A2A” is Diabetic+2A2 group, and “C+A2A” is Control+2A2 group.

FIG. 3A-B—show bar charts illustrating differences between treatments. FIG. 3A is a bar chart showing the wound size after ten days, with wound size represented as a normalized % of initial wound size. FIG. 3B is a bar chart showing the average number of days it took for the mice to achieve 95% healing in each experimental group.

FIG. 4 shows the average rate of wound healing in each experimental group over time The Y axis indicates the size of the wound, with the starting size of each wound normalized as 1. The X axis indicates number of days.

FIG. 5A-D—are a bar charts showing the average number of days it took for the mice to achieve 25%, 50%, 75%, and 90% healing, respectively for each of the experimental groups.

### SUMMARY

In one aspect, the present disclosure provides methods of treating or preventing a wound in a subject in need thereof comprising administering an anti-ceramide antibody or antigen-binding fragment thereof to the subject.

In one aspect, the present disclosure provides methods of enhancing healing of a wound in a subject in need thereof, comprising administering an anti-ceramide antibody or antigen-binding fragment thereof to the subject.

In some embodiments, the wound is a chronic wound or a diabetic wound. In some embodiments, the wound is a chronic wound. In some embodiments, the wound is a diabetic wound.

In some embodiments, the route of administration is selected from the group consisting of topical administration, intralesional administration, subcutaneous administration, transdermal administration, intramuscular administration, intravenous administration, and parenteral administration. In some embodiments, the route of administration is topical administration. In some embodiments, the route of administration is intravenous administration.

In some embodiments, the anti-ceramide antibody or antigen-binding fragment thereof is administered as a single dose. In some embodiments, the anti-ceramide antibody or antigen-binding fragment thereof is administered in two or more doses. In some embodiments, administrations of consecutive doses are separated by at least 1 day, at least 2 days, at least 3 days, at least 4 days, at least 5 days, or at least a week. In some embodiments, the duration of the administration is at least one week, at least two weeks, at least three weeks, or at least four weeks.

In some embodiments, the anti-ceramide antibody or antigen-binding fragment thereof is administered during the inflammatory stage of wound healing. In some embodiments, the anti-ceramide antibody or antigen-binding fragment thereof is administered during the proliferative stage of wound healing. In some embodiments, the anti-ceramide antibody or antigen-binding fragment thereof is administered during the remodeling stage of wound healing.

In some embodiments, the anti-ceramide antibody or antigen-binding fragment thereof is an antibody. In some embodiments, the anti-ceramide antibody or antigen-binding fragment thereof is a single-chain variable fragment (scFv).

In some embodiments, the method enhances wound healing by at least 10%, at least 20%, at least 30%, at least 50%, or at least 70% as compared to a control wound. In some embodiments, the enhancement of wound healing is measured by the decease of overall surface area of the wound at day 10, 20 or 30 post wounding. In some embodiments, the enhancement of wound healing is measured at day 10 post wounding. In some embodiments, the enhancement of wound healing is measured by the decease of time it takes to achieve 50%, 70%, 90%, 95% or 100% decrease of overall surface area of the wound. In some embodiments, the enhancement of wound healing is measured at 95% decrease of overall surface area of the wound.

In some embodiments, the anti-ceramide antibody or antigen-binding fragment thereof is administered before the onset of one or more symptoms of the wound. In some embodiments, the anti-ceramide antibody or antigen-binding fragment thereof is administered after the onset of one or more symptoms of the wound.

In some embodiments, the anti-ceramide antibody or antigen-binding fragment thereof comprises a variable heavy chain (V_{H}) and a variable light chain (V_{L}), wherein the V_{H} comprises a heavy chain complementarity determining region 1 (HCDR1) comprising the amino acid sequence of GYTFTDHTIH (SEQ ID NO: 1), an HCDR2 comprising the amino acid sequence of YNYPRDGSTKYNEKFKG (SEQ ID NO: 2), and an HCDR3 comprising the amino acid sequence of GFITTVVPSAY (SEQ ID NO: 3), and wherein the V_{L} comprises a light chain complementarity determining region 1 (LCDR1) comprising the amino acid sequence of RASKSISKYLA (SEQ ID NO: 4), an LCDR2 comprising the amino acid sequence of SGSTLQS (SEQ ID NO: 5), and an LCDR3 comprising the amino acid sequence of QQHNEYPWT (SEQ ID NO: 6). In some embodiments, the V_{H} comprises the amino acid sequence of SEQ ID NO: 7 and wherein the V_{L} comprises the amino acid sequence of SEQ ID NO: 8. In some embodiments, the anti-ceramide antibody or antigen-binding fragment thereof is a 6B5 antibody. In some embodiments, the anti-ceramide antibody or antigen-binding fragment thereof is a 6B5 scFv.

In some embodiments, the anti-ceramide antibody or antigen-binding fragment thereof comprises a variable heavy chain (V_{H}) and a variable light chain (V_{L}), wherein the V_{H} comprises a heavy chain complementarity determining region 1 (HCDR1) comprising the amino acid sequence of NYWMH (SEQ ID NO: 33), an HCDR2 comprising the amino acid sequence of AIYPGDSDTSYNQKFKG (SEQ ID NO: 34), and an HCDR3 comprising the amino acid sequence of LYYGYD (SEQ ID NO: 35), and wherein the V_{L} comprises a light chain complementarity determining region 1 (LCDR1) comprising the amino acid sequence of KSSQSLIDSDGKTFLN (SEQ ID NO: 36), an LCDR2 comprising the amino acid sequence of LVSKLDS (SEQ ID NO: 37), and an LCDR3 comprising the amino acid sequence of WQGTHFPYT (SEQ ID NO: 38). In some embodiments, the V_{H} comprises the amino acid sequence of SEQ ID NO: 39 and wherein the V_{L} comprises the amino acid sequence of SEQ ID NO: 40. In some embodiments, the anti-ceramide antibody or antigen-binding fragment thereof is a 2A2 antibody. In some embodiments, the anti-ceramide antibody or antigen-binding fragment thereof is a 2A2 scFv.

In some embodiments, the anti-ceramide antibody or antigen-binding fragment thereof comprises a variable heavy chain (V_{H}) and a variable light chain (V_{L}), wherein the V_{H} comprises a heavy chain complementarity determining region 1 (HCDR1) comprising or consisting of an amino acid sequence selected from SEQ ID NOS: 1 and 43, an HCDR2 comprising or consisting of an amino acid sequence selected from SEQ ID NOS: 44-47, and an HCDR3 comprising or consisting of an amino acid sequence of GFITTVVPSAY (SEQ ID NO: 3), and wherein the V_{L} comprises a light chain complementarity determining region 1 (LCDR1) comprising or consisting of an amino acid sequence of RASKSISKYLA (SEQ ID NO: 4), an LCDR2 comprising or consisting of an amino acid sequence of SGSTLQS (SEQ ID NO: 5), and an LCDR3 comprising or consisting of an amino acid sequence of QQHNEYPWT (SEQ ID NO: 6). In some embodiments, the HCDR1 comprises or consists of the amino acid sequence of GYTFTDHTIH (SEQ ID NO: 1), and the HCDR2 comprises or consists of the amino acid sequence of YNYPRDGSTKYNEKFQG (SEQ ID NO: 44). In some embodiments, the HCDR1 comprises or consists of the amino acid sequence of GYTFTDHTIH (SEQ ID NO: 1), and the HCDR2 comprises or consists of the amino acid sequence of YNYPREGSTKYNEKFQG (SEQ ID NO: 45). In some embodiments, the HCDR1 comprises or consists of the amino acid sequence of GYTFTDHTIH (SEQ ID NO: 1), and the HCDR2 comprises or consists of the amino acid sequence of YNYPRDVSTKYNEKFQG (SEQ ID NO: 46). In some embodiments, the HCDR1 comprises or consists of the amino acid sequence of GYTFTDHTIH (SEQ ID NO: 1), and the HCDR2 comprises or consists of the amino acid sequence of YNYPRDGSTKYAEKFQG (SEQ ID NO: 47). In some embodiments, the HCDR1 comprises or consists of the amino acid sequence of GYTFTDHTMH (SEQ ID NO: 43), and the HCDR2 comprises or consists of the amino acid sequence of YNYPRDGSTKYNEKFQG (SEQ ID NO: 44). In some embodiments, the HCDR1 comprises or consists of the amino acid sequence of GYTFTDHTMH (SEQ ID NO: 43), and the HCDR2 comprises or consists of the amino acid sequence of YNYPREGSTKYNEKFQG (SEQ ID NO: 45). In some embodiments, the HCDR1 comprises or consists of the amino acid sequence of GYTFTDHTMH (SEQ ID NO: 43), and the HCDR2 comprises or consists of the amino acid sequence of YNYPRDVSTKYNEKFQG (SEQ ID NO: 46). In some embodiments, the HCDR1 comprises or consists of the amino acid sequence of GYTFTDHTMH (SEQ ID NO: 43), and the HCDR2 comprises or consists of the amino acid sequence of YNYPRDGSTKYAEKFQG (SEQ ID NO: 47). In some embodiments, the VH comprises or consists of an amino acid sequence that is at least 90%, at least 95%, at least 97% identical, or 100% identical to SEQ ID NO: 48 and the VL comprises or consists of an amino acid sequence that is at least 90%, at least 95%, at least 97% identical, or 100% identical to SEQ ID NO: 53. In some embodiments, the VH comprises or consists of an amino acid sequence that is at least 90%, at least 95%, at least 97% identical, or 100% identical to SEQ ID NO: 48 and the VL comprises or consists of an amino acid sequence that is at least 90%, at least 95%, at least 97% identical, or 100% identical to SEQ ID NO: 54. In some embodiments, the VH comprises or consists of an amino acid sequence that is at least 90%, at least 95%, at least 97% identical, or 100% identical to SEQ ID NO: 48 and the VL comprises or consists of an amino acid sequence that is at least 90%, at least 95%, at least 97% identical, or 100% identical to SEQ ID NO: 55. In some embodiments, the VH comprises or consists of an amino acid sequence that is at least 90%, at least 95%, at least 97% identical, or 100% identical to SEQ ID NO: 49 and the VL comprises or consists of an amino acid sequence that is at least 90%, at least 95%, at least 97% identical, or 100% identical to SEQ ID NO: 53. In some embodiments, the VH comprises or consists of an amino acid sequence that is at least 90%, at least 95%, at least 97% identical, or 100% identical to SEQ ID NO: 49 and the VL comprises or consists of an amino acid sequence that is at least 90%, at least 95%, at least 97% identical, or 100% identical to SEQ ID NO: 54. In some embodiments, the VH comprises or consists of an amino acid sequence that is at least 90%, at least 95%, at least 97% identical, or 100% identical to SEQ ID NO: 49 and the VL comprises or consists of an amino acid sequence that is at least 90%, at least 95%, at least 97% identical, or 100% identical to SEQ ID NO: 55. In some embodiments, the VH comprises or consists of an amino acid sequence that is at least 90%, at least 95%, at least 97% identical, or 100% identical to SEQ ID NO: 50 and the VL comprises or consists of an amino acid sequence that is at least 90%, at least 95%, at least 97% identical, or 100% identical to SEQ ID NO: 53. In some embodiments, the VH comprises or consists of an amino acid sequence that is at least 90%, at least 95%, at least 97% identical, or 100% identical to SEQ ID NO: 50 and the VL comprises or consists of an amino acid sequence that is at least 90%, at least 95%, at least 97% identical, or 100% identical to SEQ ID NO: 54. In some embodiments, the VH comprises or consists of an amino acid sequence that is at least 90%, at least 95%, at least 97% identical, or 100% identical to SEQ ID NO: 50 and the VL comprises or consists of an amino acid sequence that is at least 90%, at least 95%, at least 97% identical, or 100% identical to SEQ ID NO: 55. In some embodiments, the VH comprises or consists of an amino acid sequence that is at least 90%, at least 95%, at least 97% identical, or 100% identical to SEQ ID NO: 51 and the VL comprises or consists of an amino acid sequence that is at least 90%, at least 95%, at least 97% identical, or 100% identical to SEQ ID NO: 53. In some embodiments, the VH comprises or consists of an amino acid sequence that is at least 90%, at least 95%, at least 97% identical, or 100% identical to SEQ ID NO: 51 and the VL comprises or consists of an amino acid sequence that is at least 90%, at least 95%, at least 97% identical, or 100% identical to SEQ ID NO: 54. In some embodiments, the VH comprises or consists of an amino acid sequence that is at least 90%, at least 95%, at least 97% identical, or 100% identical to SEQ ID NO: 51 and the VL comprises or consists of an amino acid sequence that is at least 90%, at least 95%, at least 97% identical, or 100% identical to SEQ ID NO: 55. In some embodiments, the VH comprises or consists of an amino acid sequence that is at least 90%, at least 95%, at least 97% identical, or 100% identical to SEQ ID NO: 52 and the VL comprises or consists of an amino acid sequence that is at least 90%, at least 95%, at least 97% identical, or 100% identical to SEQ ID NO: 53. In some embodiments, the VH comprises or consists of an amino acid sequence that is at least 90%, at least 95%, at least 97% identical, or 100% identical to SEQ ID NO: 52 and the VL comprises or consists of an amino acid sequence that is at least 90%, at least 95%, at least 97% identical, or 100% identical to SEQ ID NO: 54. In some embodiments, the VH comprises or consists of an amino acid sequence that is at least 90%, at least 95%, at least 97% identical, or 100% identical to SEQ ID NO: 52 and the VL comprises or consists of an amino acid sequence that is at least 90%, at least 95%, at least 97% identical, or 100% identical to SEQ ID NO: 55.

In some embodiments, the anti-ceramide antibody or antigen-binding fragment thereof is a humanized 6B5 (h6B5) antibody. In some embodiments, the anti-ceramide antibody or antigen-binding fragment thereof is a h6B5 scFv.

In some embodiments, the anti-ceramide antibody or antigen-binding fragment thereof comprises a variable heavy chain (V_{H}) and a variable light chain (V_{L}), wherein the V_{H} comprises an amino acid sequence of SEQ ID NO: 48, and wherein the V_{L} comprises an amino acid sequence of SEQ ID NO: 53. In some embodiments, the anti-ceramide antibody or antigen-binding fragment thereof comprises a variable heavy chain (V_{H}) and a variable light chain (V_{L}), wherein the V_{H} comprises an amino acid sequence of SEQ ID NO: 48, and wherein the V_{L} comprises an amino acid sequence of SEQ ID NO: 55. In some embodiments, the anti-ceramide antibody or antigen-binding fragment thereof comprises a variable heavy chain (V_{H}) and a variable light chain (V_{L}), wherein the V_{H} comprises an amino acid sequence of SEQ ID NO: 49, and wherein the V_{L} comprises an amino acid sequence of SEQ ID NO: 53. In some embodiments, the anti-ceramide antibody or antigen-binding fragment thereof comprises a variable heavy chain (V_{H}) and a variable light chain (V_{L}), wherein the V_{H} comprises an amino acid sequence of SEQ ID NO: 49, and wherein the V_{L} comprises an amino acid sequence of SEQ ID NO: 54. In some embodiments, the anti-ceramide antibody or antigen-binding fragment thereof comprises a variable heavy chain (V_{H}) and a variable light chain (V_{L}), wherein the V_{H} comprises an amino acid sequence of SEQ ID NO: 50, and wherein the V_{L} comprises an amino acid sequence of SEQ ID NO: 53. In some embodiments, the anti-ceramide antibody or antigen-binding fragment thereof comprises a variable heavy chain (V_{H}) and a variable light chain (V_{L}), wherein the V_{H} comprises an amino acid sequence of SEQ ID NO: 50, and wherein the V_{L} comprises an amino acid sequence of SEQ ID NO: 54. In some embodiments, the anti-ceramide antibody or antigen-binding fragment thereof comprises a variable heavy chain (V_{H}) and a variable light chain (V_{L}), wherein the V_{H} comprises an amino acid sequence of SEQ ID NO: 51, and wherein the V_{L} comprises an amino acid sequence of SEQ ID NO: 53. In some embodiments, the anti-ceramide antibody or antigen-binding fragment thereof comprises a variable heavy chain (V_{H}) and a variable light chain (V_{L}), wherein the V_{H} comprises an amino acid sequence of SEQ ID NO: 52, and wherein the V_{L} comprises an amino acid sequence of SEQ ID NO: 53. In some embodiments, the anti-ceramide antibody or antigen-binding fragment thereof is a humanized antibody. In some embodiments, the anti-ceramide antibody or antigen-binding fragment thereof is a humanized scFv.

### DETAILED DESCRIPTION

### Overview

The present disclosure relates to compositions and methods for enhancing wound healing. In some embodiments, provided are compositions of anti-ceramide antibodies and antigen-binding fragments thereof (e.g., scFvs) and methods of use in the treatment or prevention of wounds. In some embodiments, the wound is a chronic wound. In some embodiments, the wound is a diabetic wound. Such compositions and methods may be used in the treatment of diabetic wounds in patients who have previously failed another treatment for diabetic wounds.

### Definitions

As used in this specification and the appended claims, the singular forms “a,” “an,” and “the” include plural references unless the content clearly dictates otherwise.

As used in this specification, the term “and/or” is used in this disclosure to mean either “and” or “or” unless indicated otherwise.

Throughout this specification, unless the context requires otherwise, the words “comprise”, or variations such as “comprises” or “comprising”, will be understood to imply the inclusion of a stated element or integer or group of elements or integers but not the exclusion of any other element or integer or group of elements or integers.

As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 10% or less in either direction (greater than or less than) of the stated reference value unless otherwise stated or otherwise evident from the context (except where such number would exceed 100% of a possible value or fall below 0% of a possible value).

The term “sample” refers to a biological composition (e.g., a cell or a portion of a tissue) that is subjected to analysis and/or modification. In some embodiments, a sample is a “primary sample” in that it is obtained directly from a subject; in some embodiments, a “sample” is the result of processing of a primary sample, for example to remove certain components and/or to isolate or purify certain components of interest.

The term “subject” includes animals, such as e.g. mammals. In some embodiments, the mammal is a primate. In some embodiments, the mammal is a human. In some embodiments, subjects are livestock such as cattle, sheep, goats, cows, swine, and the like; or domesticated animals such as dogs and cats. In some embodiments (e.g., particularly in research contexts) subjects are rodents (e.g., mice, rats, hamsters), rabbits, primates, or swine such as inbred pigs and the like. The terms “subject” and “patient” are used interchangeably herein. In some embodiments, the subject may be a neonate, a juvenile, or an adult. Of particular interest are mammalian subjects. Mammalian species that may be treated with the present methods include canines and felines; equines; bovines; ovines; etc. and primates, particularly humans. Animal models, particularly small mammals (e.g. mice, rats, guinea pigs, hamsters, rabbits, etc.) may be used for experimental investigations.

As used herein, the terms “treatment,” “treating,” or “ameliorating” refers to either a therapeutic treatment or prophylactic/preventative treatment. A treatment is therapeutic if at least one symptom of disease in an individual receiving treatment improves or a treatment can delay worsening of a progressive disease in an individual or prevent onset of additional associated diseases.

As used herein, the term “effective amount” refers to the amount of an agent or composition required to result in a particular physiological effect. The effective amount of a particular agent may be represented in a variety of ways based on the nature of the agent, such as mass/volume, # of cells/volume, particles/volume, (mass of the agent)/(mass of the subject), # of cells/(mass of subject), or particles/(mass of subject). The effective amount of a particular agent may also be expressed as the half-maximal effective concentration (EC₅₀), which refers to the concentration of an agent that results in a magnitude of a particular physiological response that is half-way between a reference level and a maximum response level.

The term “antibody” refers to an immunoglobulin (Ig) molecule capable of binding to a specific target, such as a carbohydrate, polynucleotide, lipid, or polypeptide, through at least one epitope recognition site located in the variable region of the Ig molecule. As used herein, the term encompasses intact polyclonal or monoclonal antibodies and antigen-binding fragments thereof. For example, a native immunoglobulin molecule is comprised of two heavy chain polypeptides and two light chain polypeptides. Each of the heavy chain polypeptides associate with a light chain polypeptide by virtue of interchain disulfide bonds between the heavy and light chain polypeptides to form two heterodimeric proteins or polypeptides (i.e., a protein comprised of two heterologous polypeptide chains). The two heterodimeric proteins then associate by virtue of additional interchain disulfide bonds between the heavy chain polypeptides to form an immunoglobulin protein or polypeptide.

The term “antigen-binding fragment” as used herein refers to a polypeptide fragment that contains at least one Complementarity-determining region (CDR) of an immunoglobulin heavy and/or light chain that binds to at least one epitope of the antigen of interest. In this regard, an antigen-binding fragment of the herein described antibodies may comprise 1, 2, 3, 4, 5, or all 6 CDRs of a variable heavy chain (VH) and variable light chain (VL) sequence from antibodies that specifically bind ceramide. Antigen-binding fragments include proteins that comprise a portion of a full length antibody, generally the antigen binding or variable region thereof, such as Fab, F(ab′)2, Fab′, Fv fragments, minibodies, diabodies, single domain antibody (dAb), single-chain variable fragments (scFv), multispecific antibodies formed from antibody fragments, and any other modified configuration of the immunoglobulin molecule that comprises an antigen-binding site or fragment of the required specificity. In certain embodiments of the disclosure, an antigen-binding fragment, rather than an intact antibody, is used to increase tissue penetration or tumor penetration. In other embodiments, antigen-binding fragments are further modified to increase serum half-life.

“Fc region” or “Fc domain” refers to a polypeptide sequence corresponding to or derived from the portion of an antibody that is capable of binding to Fc receptors on cells and/or the C1q component of complement, thereby mediating the effector function of an antibody. Fc stands for “fragment crystalline,” the fragment of an antibody that will readily form a protein crystal. Distinct protein fragments, which were originally described by proteolytic digestion, can define the overall general structure of an immunoglobulin protein. As originally defined in the literature, the Fc region is a homodimeric protein comprising two polypeptides that are associated by disulfide bonds, and each comprising a hinge region, a CH2 domain, and a CH3 domain. However, more recently the term has been applied to the single chain monomer component consisting of CH3, CH2, and at least a portion of the hinge sufficient to form a disulfide-linked dimer with a second such chain. As such, and depending on the context, use of the terms “Fc region” or “Fc domain” will refer herein to either the dimeric form or the individual monomers that associate to form the dimeric protein. For a review of immunoglobulin structure and function, see Putnam, The Plasma Proteins, Vol. V (Academic Press, Inc., 1987), pp. 49-140; and Padlan, Mol. Immunol. 31:169-217, 1994. As used herein, the term Fc domain includes variants of naturally occurring sequences.

The term “immunoglobulin constant region” or “constant region” refers to a peptide or polypeptide sequence that corresponds to or is derived from part or all of one or more constant domains of an immunoglobulin (e.g., CH1, CH2, CH3). In certain embodiments, the constant region does not comprise a CH1 domain. In certain embodiments, the constant domains making up the constant region are human

The terms “light chain variable region” (also referred to as “light chain variable domain” or “VL”) and “heavy chain variable region” (also referred to as “heavy chain variable domain” or “VH”) refer to the variable binding region from an antibody light and heavy chain, respectively. The variable binding regions are made up of discrete, well-defined sub-regions known as “complementarity determining regions” (CDRs) and “framework regions” (FRs).

The term “immunoglobulin light chain constant region” (also referred to as “light chain constant region” or “CL”) is a constant region from an antibody light chain.

The term “immunoglobulin heavy chain constant region” (also referred to as “heavy chain constant region” or “CH”) refers to the constant region from the antibody heavy chain. The CH is further divisible, depending on the antibody isotype into CH1, CH2, and CH3 (IgA, IgD, IgG), or CH1, CH2, CH3, and CH4 domains (IgE, IgM).

The term “F(ab)” refers to two of the protein fragments resulting from proteolytic cleavage of IgG molecules by the enzyme papain. Each F(ab) comprises a covalent heterodimer of the VH chain and VL chain and includes an intact antigen-binding site. Each F(ab) is a monovalent antigen-binding fragment. The term “Fab′” refers to a fragment derived from F(ab′)2 and may contain a small portion of Fc. Each Fab′ fragment is a monovalent antigen-binding fragment.

The term “F(ab′)2” refers to a protein fragment of IgG generated by proteolytic cleavage by the enzyme pepsin. Each F(ab′)2 fragment comprises two F(ab′) fragments and is therefore a bivalent antigen-binding fragment.

An “Fd fragment” comprises the VH and CH1 domains.

An “Fv fragment” refers to a non-covalent VH::VL heterodimer which includes an antigen-binding site that retains much of the antigen recognition and binding capabilities of the native antibody molecule, but lacks the CH1 and CL domains contained within a Fab. Inbar et al. (1972) Proc. Nat. Acad. Sci. USA 69:2659-2662; Hochman et al. (1976) Biochem 15:2706-2710; and Ehrlich et al. (1980) Biochem 19:4091-4096. In some embodiments, the Fv fragment can be produced by preferential proteolytic cleavage of an IgM, and on rare occasions of an IgG or IgA immunoglobulin molecule. Fv fragments are, however, more commonly derived using recombinant techniques known in the art.

A “dAb fragment” (Ward et al., Nature 341:544 546, 1989) comprises a VH domain.

A “single-chain antibody” or an “scFv” is a fusion protein of the variable regions of the heavy (VH) and light chains (VL) of immunoglobulins, connected with a short linker peptide of ten to about 25 amino acids. The linker may be rich in glycine for flexibility, as well as serine or threonine for solubility, and can either connect the N-terminus of the VH with the C-terminus of the VL, or vice versa. The scFv retains the specificity of the original immunoglobulin, despite removal of the constant regions and the introduction of the linker. In the present disclosure, any mention of antibodies or antibody fragments or the use thereof is intended to comprise scFv molecules and the use thereof.

“Minibodies” refer to a fusion protein comprising an scFv joined to a CH3 domain and are also included herein (S. Hu et al., Cancer Res., 56, 3055-3061, 1996). See e.g., Ward, E. S. et al., Nature 341, 544-546 (1989); Bird et al., Science, 242, 423-426, 1988; Huston et al., PNAS USA, 85, 5879-5883, 1988); PCT/US92/09965; WO94/13804; P. Holliger et al., Proc. Natl. Acad. Sci. USA 90 6444-6448, 1993; Y. Reiter et al., Nature Biotech, 14, 1239-1245, 1996; S. Hu et al., Cancer Res., 56, 3055-3061, 1996.

The term “diabody” refers to a bispecific antibody in which VH and VL domains are expressed in a single polypeptide chain using a linker that is too short to allow for pairing between the two domains on the same chain, thereby forcing the domains to pair with complementary domains of another chain and creating two antigen-binding sites (see, e.g., Holliger et al., Proc. Natl. Acad. Sci. USA 90:6444-48 (1993) and Poljak et al., Structure 2:1121-23 (1994)).

The term “nanobody” or a “single domain antibody” refers to an antigen-binding fragment consisting of a single monomeric variable antibody domain. The Nanoclone method is a method for generating Nanobodies against a desired target based on automated high-throughput selection of B-cells. (See, WO 2006/079372)

The term “monoclonal antibody” as used herein refers to an antibody obtained from a population of substantially homogeneous antibodies, i.e., the individual antibodies comprising the population are identical except for possible naturally occurring mutations that may be present in minor amounts.

The term “chimeric antibody” as used herein refers to a monoclonal antibody in which a portion of the heavy and/or light chain is identical or homologous to corresponding sequences in antibodies derived from a particular species or belonging to a particular antibody class or subclass, while the remainder of the chain(s) is identical or homologous to corresponding sequences in antibodies derived from another species or belonging to another antibody class or subclass, as well as fragments of such antibodies, so long as they exhibit the desired biological activity.

The term “single chain variable fragment” or “scFv” refers to a fusion protein of the variable regions of the heavy (VH) and light chains (VL) of immunoglobulins, connected with a short linker peptide of ten to about 25 amino acids. Huston et al. (1988) Proc. Nat. Acad. Sci. USA 85(16):5879-5883. The linker can connect the N-terminus of the VH with the C-terminus of the VL, or vice versa. A number of methods have been described to discern chemical structures for converting the naturally aggregated—but chemically separated—light and heavy polypeptide chains from an antibody V region into an scFv molecule which will fold into a three dimensional structure substantially similar to the structure of an antigen-binding site. See, e.g., U.S. Pat. Nos. 5,091,513 and 5,132,405, to Huston et al.; and U.S. Pat. No. 4,946,778, to Ladner et al.

As used herein, the term “CDR” refers to the “complementarity determining region” of an immunoglobulin (antibody) molecule. CDRs are part of the variable domain in an antibody where the antibody binds to its specific antigen. There are three CDR per variable domain (i.e., CDR1, CDR2 and CDR3 in the variable domain of the light chain and CDR1, CDR2 and CDR3 in the variable domain of the heavy chain). Within the variable domain, CDR1 and CDR2 are found in the variable (V) region of a polypeptide chain, CDR3 shows the greatest variability as it is encoded by a recombination of the VJ in the case of a light chain region and VDJ in the case of heavy chain regions.

An “isolated antibody” is an antibody that (1) is not associated with naturally-associated components, including other naturally-associated antibodies, that accompany it in its native state, (2) is free of other proteins from the same species, (3) is expressed by a cell from a different species, or (4) does not occur in nature.

The term “human antibody” includes all antibodies that have one or more variable and constant regions derived from human immunoglobulin sequences. In a preferred embodiment, all of the variable and constant domains are derived from human immunoglobulin sequences (a fully human antibody). These antibodies may be prepared in a variety of ways, as described below.

As used herein, the term “humanized” refers to an antibody or antigen-binding fragment thereof derived from a non-human species that retains the antigen-binding properties of the original non-human antibody. In some embodiments, the binding fragments of an antibody (e.g., light and heavy chain variable regions, Fab, scFv) are humanized. Non-human antigen-binding fragments can be humanized using techniques known as CDR grafting (Jones et al., Nature 321:522 (1986)) and variants thereof, including “reshaping” (Verhoeyen, et al., 1988 Science 239:1534-1536; Riechmann, et al., 1988 Nature 332:323-337; Tempest, et al., Bio/Technol 1991 9:266-271), “hyperchimerization” (Queen, et al., 1989 Proc Natl Acad Sci USA 86:10029-10033; Co, et al., 1991 Proc Natl Acad Sci USA 88:2869-2873; Co, et al., 1992 J Immunol 148:1149-1154), and “veneering” (Mark, et al., “Derivation of therapeutically active humanized and veneered anti-CD18 antibodies.” In: Metcalf BW, Dalton BJ, eds. Cellular adhesion: molecular definition to therapeutic potential. New York: Plenum Press, 1994: 291-312). If derived from a non-human source, other regions of the antibody, such as the hinge region and constant region domains, can also be humanized.

As used herein, the term “pharmaceutically acceptable” refers to molecular entities and compositions that do not generally produce allergic or other serious adverse reactions when administered using routes well known in the art. Molecular entities and compositions approved by a regulatory agency of the Federal or a state government or listed in the U.S. Pharmacopeia or other generally recognized pharmacopeia for use in animals, and more particularly in humans are considered to be “pharmaceutically acceptable.”

The terms “prevent,” “prophylaxis,” and “prophylactically” refer to the administration of a compound, e.g. an anti-ceramide antibody or antigen-binding fragment thereof prior to the onset of disease (e.g., prior to the onset of certain symptoms of a disease). Preventing disease may include reducing the likelihood that the disease will occur, delaying onset of the disease, ameliorating long term symptoms, or delaying eventual progression of the disease.

Herein, the term “specifically binds” refers to the ability of an antibody or antigen-binding fragment thereof to bind a target antigen with a binding affinity (Ka) of at least 10⁵ M⁻¹ while not significantly binding other components or antigens present in a mixture. Reference to an anti-ceramide antibody herein refers to an antibody or antigen-binding fragment thereof that specifically binds to ceramide.

As used herein, the term “sequence identity” refers to a relationship between two or more polynucleotide sequences or between two or more polypeptide sequences. When a position in one sequence is occupied by the same nucleic acid base or amino acid residue in the corresponding position of the comparator sequence, the sequences are said to be “identical” at that position. The percentage sequence identity is calculated by determining the number of positions at which the identical nucleic acid base or amino acid residue occurs in both sequences to yield the number of identical positions. The number of identical positions is then divided by the total number of positions in the comparison window and multiplied by 100 to yield the percentage of sequence identity. Percentage of sequence identity is determined by comparing two optimally aligned sequences over a comparison window. The comparison window for polynucleotide sequences can be, for instance, at least 20, 30, 40, 50, 60, 70, 80, 90, 100, 110, 120, 130, 140, 150, 160, 170, 180, 190, 200, 300, 400, 500, 600, 700, 800, 900 or 1000 or more nucleic acids in length. The comparison window for polypeptide sequences can be, for instance, at least 20, 30, 40, 50, 60, 70, 80, 90, 100, 110, 120, 130, 140, 150, 160, 170, 180, 190, 200, 300 or more amino acids in length. In order to optimally align sequences for comparison, the portion of a polynucleotide or polypeptide sequence in the comparison window can comprise additions or deletions termed gaps while the reference sequence is kept constant. An optimal alignment is that alignment which, even with gaps, produces the greatest possible number of “identical” positions between the reference and comparator sequences. Percentage “sequence identity” between two sequences can be determined using the version of the program “BLAST 2 Sequences” which was available from the National Center for Biotechnology Information as of Sep. 1, 2004, which program incorporates the programs BLASTN (for nucleotide sequence comparison) and BLASTP (for polypeptide sequence comparison), which programs are based on the algorithm of Karlin and Altschul (Proc. Natl. Acad. Sci. USA 90(12):5873-5877, 1993). When utilizing “BLAST 2 Sequences,” parameters that were default parameters as of Sep. 1, 2004, can be used for word size (3), open gap penalty (11), extension gap penalty (1), gap dropoff (50), expect value (10) and any other required parameter including but not limited to matrix option. Two nucleotide or amino acid sequences are considered to have “substantially similar sequence identity” or “substantial sequence identity” if the two sequences have at least 80%, at least 85%, at least 90%, at least 95%, at least 96%, at least 97%, at least 98%, or at least 99% sequence identity relative to each other.

“Angiogenesis” refers to the formation of new blood vessels.

As used herein, the term “wound” refers to an injury to a tissue, including but not limited to, acute, subacute, delayed or difficult to heal wounds, and chronic wounds. The injury may be from trauma, violence, accident, surgery, disease. A wound may occur due to laceration or breaking of a membrane (such as the skin) and usually damage to underlying tissues. A wound may be caused by pressure sores from extended bed rest. Chronic wounds may be caused by diseases, including but not limited to diabetes; diseases of internal organs, including but not limited to diseases of the liver, kidneys, or lungs; cancer; or any other condition that slows the healing process. In some embodiments, a wound may be caused by a combination of the factors described in this paragraph. A wound may occur in a topical location or internally. A wound may be open or closed wound. Wounds include, for example, diabetic wounds or ulcers, burns, incisions, excisions, lacerations, abrasions, puncture or penetrating wounds, surgical wounds, contusions, hematomas, crushing injuries, and ulcers.

The term “diabetic wound” refers to a wound arising in a diabetic subject at least in part due to the diabetic condition, for example type I or type II diabetes. Diabetic wounds often occur in the lower limbs (e.g., diabetic foot wounds).

The terms “enhancement of wound healing” or “acceleration of wound healing” are used interchangeably and refer to improvement of wound healing process in the treatment group as compared to a control group.

### Anti-Ceramide Antibodies, Antibody Fragments, and Derivatives

The present disclosure relates to anti-ceramide antibodies and antigen-binding fragments thereof for the enhancement of wound healing, or for treating or preventing a wound. In some embodiments, the wound is a chronic wound. In some embodiments, the wound is a diabetic wound.

Ceramides are a family of waxy lipid molecules. A ceramide is composed of sphingosine and a fatty acid. Ceramides are found in high concentrations within the cell membrane of eukaryotic cells, since they are component lipids that make up sphingomyelin, one of the major lipids in the lipid bilayer. Ceramide participates in a variety of cellular signaling, including regulating differentiation, proliferation, and programmed cell death (PCD) of cells. As a bioactive lipid, ceramide has been implicated in a variety of physiological functions including apoptosis, cell growth arrest, differentiation, cell senescence, cell migration and adhesion. Roles for ceramide and its downstream metabolites have also been suggested in a number of pathological states including cancer, neurodegeneration, diabetes, microbial pathogenesis, obesity, and inflammation.

Sequences and properties of exemplary anti-ceramide antibodies are also disclosed in U.S. Pat. Pub. No. 2010/0239572 and 2017/0335014, each of which are hereby incorporated by reference. Sequences of illustrative anti-ceramide antibodies are provided in Table 1. However, any anti-ceramide antibody or antigen-binding fragment thereof may be employed according to the disclosed methods and uses.

TABLE 1
Illustrative Anti-Ceramide Antibody Sequences

| | | | SEQ |
|---|---|---|---|
| Antibody | Component | Sequence | ID |
| h6B5/m6B5 | HCDR1 | GYTFTDHTIH | 1 |
| h6B5 | HCDR1 (IH-MH) | GYTFTDHTMH | 43 |
| m6B5 | HCDR2 | YNYPRDGSTKYNEKFKG | 2 |
| h6B5 | HCDR2 (DG) | YNYPRDGSTKYNEKFQG | 44 |
| h6B5 | HCDR2 (DG-EG) | YNYPREGSTKYNEKFQG | 45 |
| h6B5 | HCDR2 (DG-DV) | YNYPRDVSTKYNEKFQG | 46 |
| h6B5 | HCDR2 (NE-AE) | YNYPRDGSTKYAEKFQG | 47 |
| h6B5/m6B5 | HCDR3 | GFITTVVPSAY | 3 |
| h6B5/m6B5 | LCDR1 | RASKSISKYLA | 4 |
| h6B5/m6B5 | LCDR2 | SGSTLQS | 5 |
| h6B5/m6B5 | LCDR3 | QQHNEYPWT | 6 |
| m6B5 | VH-0 | QVQLQQSDAELVKPGASVKISCKVSGYTFTDHTIHWM | 7 |
| | | KQRPEQGLEWIGYNYPRDGSTKYNEKFKGKATLTADK | |
| | | SSSTAYMQLNSLTSEDSAVYFCAKGFITTVVPSAYWG | |
| | | QGTLVTVSA | |
| h6B5 | VH-1 | QVQLVQSGAEVKKPGASVKVSCKASGYTFTDHTMHWV | 48 |
| | | RQAPGQGLEWMGYNYPRDGSTKYAEKFQGRVTMTADK | |
| | | STSTVYMELSSLRSEDTAVYYCAKGFITTVVPSAYWG | |
| | | QGTLVTVSS | |
| h6B5 | VH-2 | QVQLVQSGAEVKKPGASVKVSCKVSGYTFTDHTIHWM | 49 |
| | | RQAPGQGLEWMGYNYPRDGSTKYNEKFQGRVTMTADK | |
| | | STSTVYMELSSLRSEDTAVYYCAKGFITTVVPSAYWG | |
| | | QGTLVTVSS | |
| h6B5 | VH-3 | QVQLVQSGAEVKKPGATVKISCKVSGYTFTDHTIHWM | 50 |
| | | QQAPGKGLEWMGYNYPRDGSTKYNEKFQGRVTITADK | |
| | | STSTAYMELSSLRSEDTAVYYCAKGFITTVVPSAYWG | |
| | | QGTLVTVSS | |
| h6B5 | VH-4 | QVQLVQSGAEVKKPGASVKVSCKVSGYTFTDHTIHWM | 51 |
| | | RQAPGQGLEWMGYNYPREGSTKYNEKFQGRVTMTADK | |
| | | STSTVYMELSSLRSEDTAVYYCAKGFITTVVPSAYWG | |
| | | QGTLVTVSS | |
| h6B5 | VH-5 | QVQLVQSGAEVKKPGASVKVSCKVSGYTFTDHTIHWM | 52 |
| | | RQAPGQGLEWMGYNYPRDVSTKYNEKFQGRVTMTADK | |
| | | STSTVYMELSSLRSEDTAVYYCAKGFITTVVPSAYWG | |
| | | QGTLVTVSS | |
| m6B5 | VL-0 | DVQITQSPSYLAASPGETITINCRASKSISKYLAWYQ | 8 |
| | | EKPGKTNKLLIYSGSTLQSGIPSRFSGSGSGTDFTLT | |
| | | ISSLEPEDFAMYYCQQHNEYPWTFGGGTKLEIK | |
| h6B5 | VL-1 | DIQLTQSPSFLSASVGDRVTITCRASKSISKYLAWYQ | 53 |
| | | QKPGKAPKLLIYSGSTLQSGVPSRFSGSGSGTEFTLT | |
| | | ISSLQPEDFATYYCQQHNEYPWTFGGGTKVEIK | |
| h6B5 | VL-2 | DVQITQSPSFLSASVGDRVTITCRASKSISKYLAWYQ | 54 |
| | | QKPGKANKLLIYSGSTLQSGVPSRFSGSGSGTDFTLT | |
| | | ISSLQPEDFATYYCQQHNEYPWTFGGGTKVEIK | |
| h6B5 | VL-3 | DVQLTQSPSSVSASVGDRVTITCRASKSISKYLAWYQ | 55 |
| | | QKPGKAPKLLIYSGSTLQSGVPSRFSGSGSGTDFTLT | |
| | | ISSLQPEDFATYYCQQHNEYPWTFGPGTKVEIK | |
| 6C8 | HCDR1 | GYAFSSYWMN | 9 |
| 6C8 | HCDR2 | QIYPGDGDTNYNGKFKG | 10 |
| 6C8 | HCDR3 | RCYYGLYFDV | 11 |
| 6C8 | LCDR1 | KASQDINRYLS | 12 |
| 6C8 | LCDR2 | RANRLVD | 13 |
| 6C8 | LCDR3 | LQYDEFPYT | 14 |
| 6C8 | VH | QVQLQQSGAELVKPGASVKISCKASGYAFSSYWMNWV | 15 |
| | | KQRPGKGLEWIGQIYPGDGDTNYNGKFKGKATLTADK | |
| | | SSSTAYMQLSSLTSEDSAVYFCTRRCYYGLYFDVWGT | |
| | | GTTVTVSS | |
| 6C8 | VL | DIKMTQSPSSRYASLGERVTITCKASQDINRYLSWFQ | 16 |
| | | QKPGKSPKTLIYRANRLVDGVPSSRFSGSGSGQDYSL | |
| | | TISSLEYEDMGIYYCLQYDEFPYTFGGGTKLEIK | |
| 7B10 | HCDR1 | GYTFTSYWMH | 17 |
| 7B10 | HCDR2 | YINPSSGYTKYNQFKD | 18 |
| 7B10 | HCDR3 | GGYYGFAY | 19 |
| 7B10 | LCDR1 | SASSSVSYMY | 20 |
| 7B10 | LCDR2 | LTSNLAS | 21 |
| 7B10 | LCDR3 | QQWSSNPLT | 22 |
| 7B10 | VH | QVQLQQSGAELAKPGASVKLSCKASGYTFTSYWMHWV | 23 |
| | | KQRPGQGLEWIGYINPSSGYTKYNQKFKDKATLTADK | |
| | | SSSTAYMQLSSLTYEDSAVYYCARGGYYGFAYWGQGT | |
| | | LVTVSA | |
| 7B10 | VL | QIVLTQSPALMSASPGEKVTMTCSASSSVSYMYWYQQ | 24 |
| | | KPRSSPKPWIYLTSNLASGVPARFSGSGSGTSYSLTI | |
| | | SSMEAEDAATYYCQQWSSNPLTFGAGTKLELK | |
| 9H10 | HCDR1 | GFSLTGYGVH | 25 |
| 9H10 | HCDR2 | VIWSGGSTDYNAAFIS | 26 |
| 9H10 | HCDR3 | NYGYDYAMDY | 27 |
| 9H10 | LCDR1 | RASQSIGTSIH | 28 |
| 9H10 | LCDR2 | YASESIS | 29 |
| 9H10 | LCDR3 | QQSNSWPFT | 30 |
| 9H10 | VH | QVQLKQSGPGVQPSSLSITCTVSGFSLTSYGVHWVRQ | 31 |
| | | SPGKGLEWLGVIWSGGSTDYNAAFISRLSISKDNSKS | |
| | | QVFFKMNSLQADDTAIYYCARNYGYDYAMDYWGQGTS | |
| | | VTVSS | |
| 9H10 | VL | DILLTQSPAILSVSPGERVSFSCRASQSIGTSIHWYQ | 32 |
| | | QRTNGSPRLLIKYASESISGIPSRFSGSGSGTDFTLS | |
| | | INSVESEDIADYYCQQSNSWPFTFGSGTKLEIK | |
| 2A2 | HCDR1 | NYWMH | 33 |
| 2A2 | HCDR2 | AIYPGDSDTSYNQKFKG | 34 |
| 2A2 | HCDR3 | LYYGYD | 35 |
| 2A2 | LCDR1 | KSSQSLIDSDGKTELN | 36 |
| 2A2 | LCDR2 | LVSKLDS | 37 |
| 2A2 | LCDR3 | WQGTHEPYT | 38 |
| murine 2A2 | VH | EVQLQQSGTVLARPGASVKMSCKASGYTFTNYWMHWV | 39 |
| | | KQRPVQGLEWIGAIYPGDSDTSYNQKFKGKAKLTAVT | |
| | | STSTAFMELSSLTNEDSAVYYCTGLYYGYDWGQGTTL | |
| | | TVSS | |
| murine 2A2 | VL | DVLMTQTPLTLSVTIGQPASISCKSSQSLIDSDGKTF | 40 |
| | | LNWLLQRPGQSPKRLIYLVSKLDSGVPDRFTGSGSGT | |
| | | DFTLKISRVEAEDLGLYYCWQGTHFPYTFGGGTKLEI | |
| | | K | |
| humanized | Heavy chain | MDWTWRVFCLLAVAPGAHSQVQLVQSGAEVKKPGASV | 41 |
| 2A2 (h2A2) | | KVSCKASGYTFTNYWMHWVRQAPGQGLEWMGAIYPGD | |
| | | SDTSYNQKFKGRVTMTRDTSTSTVYMELSSLRSEDTA | |
| | | VYYCARLYYGYDWGQGTTVTVSSASTKGPSVFPLAPS | |
| | | SKSTSGGTAALGCLVKDYFPEPVTVSWNSGALTSGVH | |
| | | TFPAVLQSSGLYSLSSVVTVPSSSLGTQTYICNVNHK | |
| | | PSNTKVDKKVEPKSCDKTHTCPPCPAPELLGGPSVFL | |
| | | FPPKPKDTLMISRTPEVTCVVVDVSHEDPEVKENWYV | |
| | | DGVEVHNAKTKPREEQYNSTYRVVSVLTVLHQDWLNG | |
| | | KEYKCKVSNKALPAPIEKTISKAKGQPREPQVYTLPP | |
| | | SRDELTKNQVSLTCLVKGFYPSDIAVEWESNGQPENN | |
| | | YKTTPPVLDSDGSFFLYSKLTVDKSRWQQGNVFSCSV | |
| | | MHEALHNHYTQKSLSLSPG | |
| humanized | Light chain | MRLPAQLLGLLMLWVPGSSGDVVMTQSPLSLPVTLGQ | 42 |
| 2A2 (h2A2) | | PASISCKSSQSLIDSDGKTFLNWFQQRPGQSPRRLIY | |
| | | LVSKLDSGVPDRFSGSGSGTDFTLKISRVEAEDVGVY | |
| | | YCWQGTHFPYTFGQGTKLEIKRTVAAPSVFIFPPSDE | |
| | | QLKSGTASVVCLLNNFYPREAKVQWKVDNALQSGNSQ | |
| | | ESVTEQDSKDSTYSLSSTLTLSKADYEKHKVYACEVT | |
| | | HQGLSSPVTKSFNRGEC | |

In some embodiments, the anti-ceramide antibody or antigen-binding fragment thereof is humanized 6B5 (h6B5). In some embodiments the h6B5 antibody or antigen-binding fragment thereof comprises an HCDR1, HCDR2, HCDR3, LCDR1, LCDR2, and LCDR3. In some embodiments, the sequences of the h6B5 antibody are provided in U.S. Provisional Application 62/991,232, filed on Mar. 18, 2020, the contents of which are incorporated by reference in its entirety for all purposes here. Sequences for each of the CDRs for the h6B5 antibody or antigen-binding fragment thereof are disclosed throughout this specification, and summarized in Table 2, below.

TABLE 2
Combinations of CDR Sequences for
h6B5 Antibodies and Antigen Binding
Fragments thereof

| | HCDR1 | HCDR2 | HCDR3 |
|---|---|---|---|
| h6B5 | GYTFTDHTIH | YNYPRDGSTKYNEKFQG | GFITTV |
| | (SEQ ID | (SEQ ID NO: 44) | VPSAY |
| | NO: 1) | YNYPREGSTKYNEKFQG | (SEQ ID |
| | GYTFTDHTMH | (SEQ ID NO: 45) | NO: 3) |
| | (SEQ ID | YNYPRDVSTKYNEKFQG | |
| | NO: 43) | (SEQ ID NO: 46) | |
| | | YNYPRDGSTKYAEKFQG | |
| | | (SEQ ID NO: 47) | |
| | LCDR1 | LCDR2 | LCDR3 |
| h6B5 | RASKSISKYLA | SGSTLQS | QQHNEYPWT |
| | (SEQ ID | (SEQ ID | (SEQ ID |
| | NO: 4) | NO: 5) | NO: 6) |

In some embodiments, the anti-ceramide antibody is selected from the group consisting of a monoclonal antibody, a chimeric antibody, a humanized antibody, a human antibody, a recombinant antibody, or a synthetic antibody. In some embodiments, the anti-ceramide antigen-binding antibody fragment is an antigen-binding fragment of any one of the foregoing. In some embodiments, the anti-ceramide antigen-binding antibody fragment is an Fab fragment, an Fab′ fragment, an F(ab′)2 fragment, an Fv fragment, an Fd fragment, a dAb fragment, a diabody, an scFv or the like. In some embodiments, the anti-ceramide antibodies and antigen-binding fragments thereof are produced using recombinant DNA technologies. Procedures for the expression and purification of recombinant proteins are well established in the art.

In some embodiments, the anti-ceramide antibody or antigen-binding fragment thereof is a single-chain variable fragment (scFv). In some embodiments, the scFv comprises the CDR sequences and/or the variable chain sequences of the 2A2, h2A2, 6C8, 7B10, 9H10, h6B5, or 6B5 antibodies.

In some embodiments, the anti-ceramide antibody or antigen-binding fragment thereof is the 2A2 antibody or an antigen-binding fragment thereof, as described in U.S. Pat. Pub. No. 2010/0239572. In some embodiments, the anti-ceramide antibody or antigen-binding fragment thereof is the 6B5 antibody or an antigen-binding fragment thereof, as described in U.S. Pat. Pub. No. 2017/0335014. In some embodiments, the anti-ceramide antibody or antigen-binding fragment thereof is the h6B5 antibody or an antigen-binding fragment thereof of the disclosure. In some embodiments, the anti-ceramide antibody or antigen-binding fragment thereof is an scFv. In some embodiments, the scFV comprises the CDR sequences of any of the antibodies disclosed in Table 1. In some embodiments, the scFv comprises the CDR sequences of 2A2. In some embodiments, the scFv comprises the CDR sequences of 6B5. In some embodiments, the scFv comprises the CDR sequences of h6B5. In some embodiments, the scFv comprises the variable heavy and light chain sequences of h2A2. In some embodiments, the scFv comprises the variable heavy and light chain sequences of 6B5. In some embodiments, the scFv comprises the variable heavy and light chain sequences of h6B5.

In some embodiments, an anti-ceramide antibody or antigen-binding fragment thereof has any immunoglobulin isotype. An immunoglobulin may be from any of the commonly known isotypes, including but not limited to IgA, secretory IgA, IgG and IgM. The IgG isotype is divided in subclasses in certain species: IgG1, IgG2, IgG3 and IgG4 in humans, and IgG1, IgG2a, IgG2b and IgG3 in mice. In some embodiments, anti-ceramide antibodies or antigen-binding fragments thereof comprise one or more modifications in the Fc region. Certain modifications can provide desired effector functions or serum half-life. In some embodiments, with the appropriate Fc regions, a naked antibody bound on the cell surface can induce cytotoxicity, e.g., via antibody-dependent cellular cytotoxicity (ADCC) or by recruiting complement in complement dependent cytotoxicity (CDC), or by recruiting nonspecific cytotoxic cells that express one or more effector ligands that recognize bound antibody on a target cell and subsequently cause phagocytosis of the target cell in antibody dependent cell-mediated phagocytosis (ADCP), or some other mechanism. Where it is desirable to eliminate or reduce effector function, so as to minimize side effects or therapeutic complications, certain other Fc regions may be used. The Fc region of antibodies can be modified to increase the binding affinity for FcRn and thus increase serum half-life. Alternatively, the Fc region can be conjugated to PEG or albumin to increase the serum half-life, or some other conjugation that results in a desired effect.

In some embodiments, the anti-ceramide antibody or antigen-binding fragment thereof comprises a detectable label or tag. Exemplary detectable labels include fluorescent tags, affinity tags, radioisotopes, luminescent markers, particulate labels, chromophores, phosphorescent markers, and enzyme labels. Exemplary fluorescent labels include GFP, RFP, and YFP. Exemplary enzyme labels include horseradish peroxidase and alkaline phosphatase. Exemplary peptide tags include His-tag, MBP, and streptavidin.

The detection means is determined by the chosen label. Appearance of the label or its reaction products can be achieved using the naked eye, in the case where the label is particulate and accumulates at appropriate levels, or using instruments such as a spectrophotometer, a luminometer, a fluorimeter, or by ELISA or Western blot.

### Wound Healing

In one aspect, the present disclosure provides methods and compositions for enhancement of wound healing. In one aspect, the present disclosure provides methods and compositions for treating or preventing a wound.

Natural wound healing occurs in clearly defined stages. Skin wounds of acute nature may heal in 1-3 weeks in a biological process that restores the integrity and function of the skin and the underlying tissue. Such wounds may be the result of a scrape, abrasion, cut, graze, incision, tear, or bruise to the skin.

Wounds may be classified into one of four grades depending on the depth of the wound: i) Grade I: wounds limited to the epithelium; ii) Grade II: wounds extending into the dermis; iii) Grade III: wounds extending into the subcutaneous tissue; and iv) Grade IV (or full-thickness wounds): wounds wherein bones are exposed (e.g., a bony pressure point such as the greater trochanter or the sacrum). The term “partial thickness wound” refers to wounds that encompass Grades I-III; examples of partial thickness wounds include pressure sores, venous stasis ulcers, and diabetic wounds or ulcers. The term “deep wound” is meant to include both Grade III and Grade IV wounds.

Natural wound healing mainly occurs according to three major chronological sequences. Each of these sequences is characterized by specific cellular activities and is controlled by a multitude of regulatory signals (both positive and negative) which collectively manage and support the progression of the repair process. Thus, the following are distinguished as:
(a) the inflammatory phase;
(b) the proliferative phase (which comprises the granulation phase and the epithelialization phase); and
(c) the remodeling phase.

The first phase, which is the inflammatory phase, begins as soon as blood vessels are burst, an event which triggers the formation of a clot (blood coagulation) that is mainly composed of fibrin and fibronectin and that will constitute a provisional matrix. This matrix in part fills the lesion and enables the migration, within the damaged area, of the inflammatory cells recruited to ensure detersion of the wound. The platelets present also release factors (for example cytokines and/or growth factors) enabling cells involved in the healing process to be recruited. This phase is characterized by infiltration and activation of numerous inflammatory cells (polymorphonuclear cells, macrophages) at the site of the lesion, which defend the organism against any foreign microorganisms and also clean or deterge the wound.

The second phase corresponds to the development of granulation tissue. First, colonization of the injury by migration and proliferation of fibroblasts is observed. Then, the migration of endothelial cells from healthy vessels allows neovascularization, or angiogenesis, of the damaged tissue. In the granulation tissue, fibroblasts are activated and differentiate into myofibroblasts with significant contractile properties provided by actin microfilaments, thus enabling wound contraction. The microfilaments are expressed via a protein, α-smooth muscle actin. These myofibroblasts play an important role in the formation and contraction of the granulation tissue which leads to healing of the lesion. Keratinocytes then migrate from the edges of the wound and then differentiate, leading to reconstruction of the epidermis. This phase of development of the granulation tissue is initiated following prior reduction in the general state of inflammation of the lesion, gradual disappearance of polymorphonuclear neutrophils and appearance of macrophages, including “repair” macrophages. This transition from the inflammatory phase to the proliferation/repair phase is known as the resolution phase of inflammation.

The third phase of the process is a remodeling stage with the goal of reconstructing a functional tissue, so that the newly formed tissue takes on the initial characteristics and properties of the original tissue. Part of the extracellular matrix is digested by proteases (essentially matrix metalloprotease and elastases), and reorganization of the extracellular matrix is observed. Type III collagen, which is predominant within the granulation tissue, is gradually replaced by type I collagen which is the main matrix component of the dermis. At the end of the maturation phase, the fibroblasts, myofibroblasts and vascular cells experience reduced proliferation and/or activity. Then, the excess cells die through apoptosis, with concomitant remodeling of the extracellular matrix.

If a wound does not heal with in the normal time period, it is considered a “chronic wound”. For example, a chronic wound may take longer than 2 weeks, longer than 3 weeks, longer than 4 weeks, longer than 5 weeks, longer than 6 weeks, longer than 6 weeks, longer than 7 weeks, longer than 8 weeks, longer than 9 weeks, longer than 10 weeks, longer than 11 weeks, longer than 12 weeks, longer than 3 months, longer than 4 months, longer than 5 months, or longer than 6 months to heal. In some embodiments, a chronic wound does not begin healing after a period of 3, 4, 5, 6, 7, 8, 9, or 10 weeks starting from the appearance of the wound.

In the case of chronic wounds, the wound may be attenuated at one of the stages of healing or fail to progress through the normal stages of healing. A chronic wound may include, for example, a wound that is characterized at least in part by one or more of: 1) a prolonged inflammatory phase, 2) a slow-forming or defective extracellular matrix (ECM), and 3) a stalled or decreased rate of epithelialization. A chronic wound may have been present for a relatively short period of time, such as a month, or it may have been present for several years. The compositions and methods described herein can initiate and enhance the healing of a chronic wound.

Chronic skin wounds include, but are not limited to, skin ulcers, bed sores, pressure sores, diabetic wounds (e.g., diabetic ulcers and sores), and other skin disorders. In some embodiments, the chronic wound is selected from a diabetic wound, a venous ulcer, an arterial ulcer, a pressure ulcer, and a vasculitic ulcer. In some embodiments, the chronic wound is a diabetic wound.

Chronic skin wounds can be any size, shape or depth, and may appear discolored as compared to normal, healthy skin pigment. Chronic skin wounds can bleed, swell, seep purulent discharge or other fluid, cause pain or cause movement of the affected area to be difficult or painful. Chronic skin wounds can become infected, producing elevated body temperatures, as well as pus or discharge that is milky, yellow, green, or brown in color. The discharge can be odorless or have a pungent odor. If infected, chronic skin wounds may be red, tender, or warm to the touch.

Chronic skin wounds can be caused by diabetes, poor blood supply, low blood oxygen, by conditions where blood flow is decreased due to low blood pressure, or by conditions characterized by occluded, blocked, or narrowed blood vessels. A low oxygen supply can be caused by certain blood, heart, and lung diseases, and/or by smoking cigarettes. Chronic skin wounds can also be the result of repeated trauma to the skin, such as swelling or increased pressure in the tissues, or constant pressure on the wound area. Chronic skin wounds can also be caused by a weakened or compromised immune system. A weakened or compromised immune system can be caused by increasing age, radiation, poor nutrition, and/or medications, such as anti-cancer medicines or steroids. Chronic skin wounds can also be cause by bacterial, viral or fungal infections, or the presence of foreign objects.

Impaired wound healing following injury in diabetic subjects represents a major clinical problem, resulting in prolonged hospitalizations and significant healthcare expenditures. Two-thirds of all non-traumatic amputations are preceded by a diabetic wound. The impaired healing of diabetic wounds is multifactorial and has been characterized by decreased production of chemokines, decreased angiogenesis, and an abnormal inflammatory response.

Without being bound any particular theory, it is contemplated that the absence of healing of these diabetic wounds is at least partially associated with an increased bioavailability of glucose. This brings about numerous physiological and metabolic alterations such as thickening of the skin, or significant oxidative stress leading to neuropathy and arteriopathy. Arteriopathy and neuropathy are two major risk factors for chronification and thus delaying of the healing of diabetic wounds, and more particularly diabetic foot wounds. The most well-known types of chronic non-diabetic wounds, such as bed sores or venous, arterial or mixed ulcers, do not follow from the same pathology. For example, venous insufficiency is the cause of the formation, chronification, and hence delayed healing of venous ulcers. Bed sores, for their part, are wounds which arise after cycles of ischemia and reperfusion following excessive and prolonged pressure and friction on cutaneous tissues.

Several major problems disrupt the correct wound healing sequence in diabetic subjects. A first delay in healing occurs from the inflammatory phase: passage from the inflammatory phase to the proliferative phase is disrupted. The inflammatory phase is an essential phase in healing, but it must be temporary. Resolution of inflammation is a critical point which conditions the initiation of the other phases of healing. This dynamic event involves the disappearance of the inflammatory cells (polymorphonuclear neutrophils) and the appearance of macrophages. Then, chemotactic and angiogenic anti-inflammatory mediators are produced, which notably enable the migration and differentiation of fibroblasts, which are key cells in the granulation phase. A disruption to this inflammatory phase, as is the case in the aforementioned subjects, causes an abnormal lengthening of the inflammatory phase and gives rise to chronicity of the wound, thereby delaying all the subsequent stages of healing. Finally, the phase of wound closure notably during epithelialization is delayed or even, in the majority of cases, does not occur.

### Role of Ceramide and ASM in Wound Healing

Without being bound by any particular theory, it is contemplated that the formation of Ceramide Rich Platform (CRP) underlies microvascular endothelial pathogenesis, which contributes to difficulty in wound healing, for example, in the cases of chronic wound or diabetic wound.

Sphingolipids represent a major component of membrane microdomains, and ceramide-enriched microdomains appear to be a prerequisite for inflammatory cytokine signaling. Acid sphingomyelinase (ASM) and neutral sphingomyelinase (NSM) are key regulatory enzymes of sphingolipid metabolism, promoting sphingomyelin hydrolysis to proinflammatory ceramide. ASM is an important early responder in inflammatory cytokine signaling.

Endothelial tissue injury leads to membrane damage and acid sphingomyelinase-dependent ceramide formation in exocellular leaflet of cell membranes. During evolution of post-injury tissue damage, CRP-dependent apoptosis likely represents a feed-forward process. In immune-mediated tissue injury, cytolytic T cells also induce CRPs on target cells which are required for efficient cell killing. Disruption of this process prevents further evolution of immune-mediated tissue injury, and lead to initiation of a tissue-reparative process that is robust and durable.

Administration of the anti-ceramide antibody or an antigen-binding fragment thereof restores the vascular endothelium homeostasis, and results in regulation of signaling between blood and tissue, trafficking of hematopoietic cells, maintenance of non-thrombogenic blood flow, facilitation of immune and inflammatory responses, and homeostatic repair and regeneration without provoking fibrosis following injury. In addition, since Ceramide Rich Platform is present mainly in injured cells, systematic delivery of an anti-ceramide antibody or antigen binding fragment thereof may act locally on injured tissue without affecting normal tissues, therefore potentially providing a wide therapeutic index.

### Pharmaceutical Compositions, Administration Routes, Dosages, and Dosing Schedules

In some embodiments, the disclosure provides a pharmaceutical composition comprising an anti-ceramide antibody or antigen-binding fragment thereof for the enhancement of wound healing, or for treating or preventing a wound.

For administration, an antibody or fragment of the present disclosure (e.g., anti-ceramide antibodies and antigen-binding fragments thereof) may be formulated as a pharmaceutical composition. A pharmaceutical composition may comprise: (i) an anti-ceramide antibody or antigen-binding fragment thereof; and (ii) a pharmaceutically acceptable carrier, diluent or excipient. A pharmaceutical composition comprising an anti-ceramide antibody or antigen-binding fragment thereof, and/or scFv can be formulated according to known methods to prepare pharmaceutically useful compositions, whereby the therapeutic molecule is combined in a mixture with a pharmaceutically acceptable carrier, diluent or excipient. Suitable carriers, diluents or excipients are well-known to those in the art. (See, e.g., Gennaro (ed.), Remington's Pharmaceutical Sciences (Mack Publishing Company, 19th ed. 1995).) Formulations can further include one or more excipients, preservatives, solubilizers, buffering agents, albumin to prevent protein loss on vial surfaces, etc.

In some embodiments, a pharmaceutical composition may be formulated in one of the following dosage forms: an oral unit dosage form, an intravenous unit dosage form, an intranasal unit dosage form, a suppository unit dosage form, an intradermal unit dosage form, an intramuscular unit dosage form, an intraperitoneal unit dosage form, a subcutaneous unit dosage form, or a topical dosage form. In some embodiments, a pharmaceutical composition may be formulated in a topical dosage form. In some embodiments, a pharmaceutical composition may be formulated in an intravenous dosage form.

In some embodiments, the pharmaceutical composition is formulated for topical administration. In some embodiments, topical pharmaceutical composition of the anti-ceramide antibody or antigen binding fragment thereof may be formulated in combination with a pharmaceutically acceptable carrier. Non-limiting examples of dosage forms for the topical composition include powders, sprays, foams, jellies, ointments, pastes, creams, lotions, gels, solutions, patches, suppositories and liposomal preparations. The dosage forms may be formulated with mucoadhesive polymers for sustained release of active ingredients. The active compound may be mixed under sterile conditions with a pharmaceutically acceptable carrier, and with any preservatives, buffers, or propellants, which may be required. Topical preparations can be prepared by combining the active ingredient with conventional pharmaceutical diluents and carriers commonly used in topical dry, liquid, cream and aerosol formulations. Ointment and creams may, for example, be formulated with an aqueous or oily base with the addition of suitable thickening and/or gelling agents. Such bases may include water and/or an oil such as liquid paraffin or a vegetable oil such as peanut oil or castor oil. Thickening agents which may be used according to the nature of the base include soft paraffin, aluminum stearate, cetostearyl alcohol, propylene glycol, polyethylene glycols, woolfat, hydrogenated lanolin, beeswax, and the like. Lotions may be formulated with an aqueous or oily base and, in general, also include one or more of the following: stabilizing agents, emulsifying agents, dispersing agents, suspending agents, thickening agents, coloring agents, perfumes, and the like. Powders may be formed with the aid of any suitable powder base, e.g., talc, lactose, starch, and the like. Drops may be formulated with an aqueous base or nonaqueous base also comprising one or more dispersing agents, suspending agents, solubilizing agents, and the like.

In some embodiments, the ointments, pastes, creams and gels also may contain excipients, such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays also can contain excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

In some embodiments, the anti-ceramide antibody or antigen binding fragment thereof can be formulated with a pharmaceutically acceptable carrier and at least one of the following second pharmacologic agents: a local anesthetic (e.g., lidocaine, prilocaine, etc.), local anti-inflammatory agent (e.g., naproxen, pramoxicam, etc.), corticosteroid (e.g., cortisone, hydrocortisone, etc.), anti-itch agent (e.g., loperamide, diphylenoxalate, etc.), an agent that interferes with the activation of peripheral sensory neurons, including divalent and trivalent metal ions (e.g., manganese, calcium, strontium, nickel, lanthanum, cerium, zinc, etc.), analgesic agents, a lubricant, yeast-based product (e.g., lyophilized yeast, yeast extract, etc.), a spermicide, growth-promoting and/or wound healing-promoting agent known to promote re-epithelialization (e.g., platelet-derived growth factor (PDGF), interleukin-11 (IL-11), etc.), anti-microbial agent (e.g., Neosporin, polymyxin B sulfate, bacitracin zinc, etc.), mucoadhesive agent (e.g., cellulose derivatives, etc.), cytoprotectant agent (e.g., colloidal bismuth, misoprostol, sucralfate, etc.) as defined in Goodman and Gilman, The Pharmacological Basis of Therapeutics, or methanol.

The anti-ceramide antibodies and antigen-binding fragments thereof described herein can be administered to subjects by one or more administration routes. Possible administration routes include, for example, by intramuscular, subcutaneous, intravenous, intra-atrial, intra-articular, parenteral, intranasal, intrapulmonary, transdermal, intrathecal, oral, topical, and intralesional routes. In some embodiments, the administration route is selected from the group consisting of topical administration, intralesional administration, subcutaneous administration, transdermal administration, intramuscular administration, intravenous administration, and parenteral administration.

In some embodiments, administration of the anti-ceramide antibodies and antigen-binding fragments thereof, or pharmaceutical compositions thereof disclosed herein comprises or consists of topical administration. In some embodiments, administration of the anti-ceramide antibodies and antigen-binding fragments thereof, or pharmaceutical compositions thereof disclosed herein comprises or consists of intralesional administration. In some embodiments, administration of the anti-ceramide antibodies and antigen-binding fragments thereof, or pharmaceutical compositions thereof disclosed herein comprises or consists of subcutaneous administration. In some embodiments, administration of the anti-ceramide antibodies and antigen-binding fragments thereof, or pharmaceutical compositions thereof disclosed herein comprises or consists of transdermal administration. In some embodiments, administration of the anti-ceramide antibodies and antigen-binding fragments thereof, or pharmaceutical compositions thereof disclosed herein comprises or consists of intramuscular administration. In some embodiments, administration of the anti-ceramide antibodies and antigen-binding fragments thereof, or pharmaceutical compositions thereof disclosed herein comprises or consists of intravenous administration. In some embodiments, administration of the anti-ceramide antibodies and antigen-binding fragments thereof, or pharmaceutical compositions thereof disclosed herein comprises or consists of parenteral administration.

For prevention and treatment purposes, anti-ceramide antibodies and antigen-binding fragments thereof can be administered to a subject in a single bolus delivery, via continuous delivery (e.g., continuous transdermal delivery) over an extended time period, or in a repeated administration protocol (e.g., on an hourly, daily, weekly, monthly, or yearly basis).

In some embodiments, the methods provided herein comprise administering a therapeutically effective dose of an anti-ceramide antibody or antigen-binding fragment thereof. A therapeutically effective dose, dosage, or amount, as defined above, refers to the amount of an anti-ceramide antibody or antigen-binding fragment thereof required to result in a particular physiological effect, e.g., prevention or amelioration of one or more symptoms of wounds, or enhancement of wound healing. Determination of therapeutically effective dosages in this context is typically based on animal model studies followed up by human clinical trials and is guided by determining effective dosages and administration protocols that significantly reduce the occurrence or severity of wounds or enhance wound healing in model subjects. Effective doses of the compositions of the present disclosure vary depending upon many different factors, including means of administration, target site, physiological state of the patient, whether the patient is human or an animal, other medications administered, whether treatment is prophylactic or therapeutic, as well as the specific activity of the composition itself and its ability to elicit the desired response in the individual. Typically, dosage regimens are adjusted to provide an optimum therapeutic response, i.e., to optimize safety and efficacy.

In some embodiments, the dose of an anti-ceramide antibody or antigen-binding fragment thereof is between about 0.1 μg to 100 mg/kg or 1 μg/kg to about 50 mg/kg, or 10 μg to 5 mg/kg′. In some embodiments, an effective amount of the anti-ceramide antibody or antigen-binding fragment thereof is between about 1 μg/kg and about 20 mg/kg, between about 10 μg/kg and about 10 mg/kg, or between about 0.1 mg/kg and about 5 mg/kg. The anti-ceramide antibodies and antigen-binding fragments thereof described herein may also be administered at a dosage from about 0.001 to about 10 milligrams (mg) per kilogram (mpk) of body weight, given as a single dose or in two or more doses. For administration to a human adult patient, the therapeutically effective amount may be administered in doses in the range of 0.2 mg to 800 mg per dose, including but not limited to 0.2 mg per dose, 0.5 mg per dose, 1 mg per dose, 5 mg per dose, 10 mg per dose, 25 mg per dose, 100 mg per dose, 200 mg per dose, and 400 mg per dose, and one or more doses may be administered in a course of treatment. In some embodiments, the total daily dosage of the anti-ceramide antibodies and antigen-binding fragments thereof described herein can range from about 1 mg to about 2 g, from about 100 mg to about 1.5 g, or from about 200 mg to about 1200 mg.

In some embodiments, the dose of an anti-ceramide antibody or antigen-binding fragment thereof is between about 0.1 μg/cm² to 100 mg/cm² or 1 μg/cm² to about 50 mg/cm², or 10 μg/cm² to 5 mg/cm² of surface area of the wound. In some embodiments, an effective amount of the anti-ceramide antibody or antigen-binding fragment thereof is between about 1 μg/cm² and about 20 mg/cm², between about 10 μg/cm² and about 10 mg/kgcm² or between about 0.1 mg/cm² and about 5 mg/cm². The anti-ceramide antibodies and antigen-binding fragments thereof described herein may also be administered at a dosage from about 0.001 to about 10 milligrams (mg) per square centimeter (cm²) of wound, given as a single dose or in two or more doses. For administration to a human adult patient, the therapeutically effective amount may be administered in doses in the range of 0.2 mg to 800 mg per dose, including but not limited to 0.2 mg per dose, 0.5 mg per dose, 1 mg per dose, 5 mg per dose, 10 mg per dose, 25 mg per dose, 100 mg per dose, 200 mg per dose, and 400 mg per dose, and one or more doses may be administered in a course of treatment. In some embodiments, the total daily dosage of the anti-ceramide antibodies and antigen-binding fragments thereof described herein can range from about 1 mg to about 2 g, from about 100 mg to about 1.5 g, or from about 200 mg to about 1200 mg.

In some embodiments, anti-ceramide antibodies, antigen-binding fragments thereof, or compositions comprising may be formulated at a concentration of about 0.1 mg/mL, 0.5 mg/mL, 1 mg/mL, 5 mg/mL, 10 mg/mL, 15 mg/mL, 20 mg/mL, 25 mg/mL, 30 mg/mL, 35 mg/mL, 40 mg/mL, 45 mg/mL, or 50 mg/mL. The concentration may be 0.1-1 mg/mL, 1-5 mg/mL, 5-10 mg/mL, or 10-50 mg/mL. In some embodiments, the disclosed antibodies, fragments or compositions may be administered in a dose of about 0.05 mg, 0.1 mg, 0.2 mg, 0.3 mg, 0.4 mg, 0.5 mg, 0.6 mg, 0.7 mg, 0.8 mg, 0.9 mg, or 1 mg. The volume of the dose may be about 0.005 mL, 0.01 mL, 0.02 mL, 0.03 mL, 0.04 mL, 0.05 mL, 0.06 mL, 0.07 mL, 0.08 mL, 0.09 mL, or 0.1 mL.

The anti-ceramide antibodies and antigen-binding fragments thereof described herein can be administered at different times of the day. In one embodiment, the dose can be administered in the evening. In another embodiment, the dose can be administered in the morning. Dosages may be administered in single or multiple administrations, including, e.g., multiple weekly, bi-weekly, monthly, or yearly administrations. In some embodiments, a single dose of anti-ceramide antibody or antibody fragment is administered to a subject in need thereof. In some embodiments, a patient may receive two or more doses of anti-ceramide antibody treatments. In some embodiments, a patient may receive two or more doses of anti-ceramide antibody treatments, wherein consecutive doses are separated by at least 1 day, at least 2 days, at least 3 days, at least 4 days, at least 5 days, at least 6 days, or at least a week. In some embodiments, a patient may receive two or more doses of anti-ceramide antibody treatments, wherein consecutive doses are separated by a period of at least one week, at least two weeks, at least three weeks, at least four weeks, at least five weeks, at least six weeks, at least seven weeks, at least eight weeks, at least one month, at least two months, or at least three months. In some embodiments, two or more doses may be administered to a patient in need thereof separated by a period of about one week to about two weeks, about two weeks to about four weeks, about one month to about two months, about two months to about four months, or about one month to about six months. In some embodiments, the duration of the administration is at least 2 days, at least 3 days, at least 4 days, at least 5 days, at least 6 days, at least one week, at least two weeks, at least three weeks, at least one month, at least two months, at least three months, at least four months, at least five months, at least six months, at least nine months, at least one year, at least two years, at least three years, at least four years, or at least five years. In some embodiments, administrations can be on an irregular basis as indicated by monitoring clinical symptoms of the disorder.

Dosage of the pharmaceutical composition comprising anti-ceramide antibodies and antigen-binding fragments thereof can be varied by the attending clinician to maintain a desired concentration at a target site. Higher or lower concentrations can be selected based on the mode of delivery. The anti-ceramide antibodies, or antigen-binding fragments thereof may be administered at any time during a subject's life. Administration may occur during the inflammatory stage, the proliferative stage, the remodeling stage, or a combination thereof, of wound healing. In some embodiments, administration occurs during the inflammatory stage of wound healing. In some embodiments, administration occurs during the proliferative stage of wound healing. In some embodiments, administration occurs during the remodeling stage of wound healing.

### Therapeutic Methods & Uses

In some embodiments, the present disclosure provides methods of treating, preventing, or ameliorating a wound in a subject, comprising administering to the subject a therapeutically effective amount of an anti-ceramide antibody or an antigen-binding fragment thereof. In some embodiments, the wound is a chronic wound. In some embodiments, the wound is a diabetic wound.

In some embodiments, the present disclosure provides methods of enhancing the healing of a wound in a subject in need thereof, comprising administering to the subject a therapeutically effective amount of anti-ceramide antibody or an antigen-binding fragment thereof. In some embodiments, the wound is a chronic wound. In some embodiments, the wound is a diabetic wound.

### Subjects

Subjects for treatment according to the methods disclosed herein include those who have or are at risk for developing a wound (e.g., a diabetic wound). Subjects who have a wound (e.g., a diabetic wound) may have early stage or late stage disease.

In some embodiments, subjects are those who have previously received one or more treatments for the wound (e.g., diabetic wound) but failed to respond to the previous treatment. In such embodiments, a “failure to respond” indicates that the previous treatment failed to ameliorate and/or improve the wound (e.g., diabetic wound). In some embodiments, the previous therapy may have shown some results, but may not have achieved the desired performance or may have stopped showing efficacy after some period of time.

### Treatment Readouts

Wound healing can be measured by a variety of means. In some embodiments, wound healing is measured by the change of overall surface area of the wound. In some embodiments, would healing is measured by the change of the length of the principal axes (length and width) of the wound. In some embodiments, wound healing is measured by the wound margin distance from the wound center. In some embodiments, wound healing is measured by the change of the perimeter of the wound. In some embodiments, wound healing is measured by the change of the surface area-to-perimeter (S/P) ratio. Assessments of these parameters can be made by a variety of methods, for example, computer-assisted planimetry. In some embodiments, the degree of wound healing is expressed by the percentage of the value over time as compared to the initial value of any one of these parameters.

Provided are methods of enhancing wound healing with an anti-ceramide antibody or antigen-binding fragment thereof. Efficacy of such treatment may be characterized, evaluated, measured, and/or monitored based on several parameters.

In some embodiments, the improvement is measured by the decrease of time to achieve a certain degree of wound healing. For example, if it takes 25 days for the wound in the control group to heal and only 20 days for the wound in the treatment group to heal, then the wound healing is enhanced/accelerated by 20% by such a measurement. In some embodiments, the improvement is measured by the increase of the relative degree of wound healing at a preset time point. For example, if at day 20 post wounding, the treatment group achieves an average 50% degree of wound healing (e.g., as measured by overall surface area) and the group only achieves an average 40% degree of wound healing, then the wound healing is enhanced/accelerated by 25% by such a measurement. A person skilled in the art will readily recognize proper control group (e.g., control wound). In some embodiments, the control wound receives identical treatment except for the anti-ceramide antibody or antigen-binding fragment thereof (e.g., those recited in the claims). In some embodiments, the control wound receives standard-of-care treatment. In all cases, references to a control group is meant to indicate that the recited property (e.g., enhanced wound healing) is the result of the use of the anti-ceramide antibody or antigen-binding fragment thereof.

In some embodiments, the method provided herein enhances wound healing in a subject, as compared to a control wound, by at least 5%, at least 10%, at least 15%, at least 20%, at least 25%, at least 30%, at least 40%, at least 50%, at least 60%, at least 70%, at least 80%, at least 90%, at least 95%, or at least 99%, including all ranges and subranges therebetween, as measured by the decrease of time it takes to improve the wound according to one of the wound healing parameters at a preset degree. In some embodiments, the method provided herein enhances wound healing by at least 10%. In some embodiments, the method provided herein enhances wound healing by at least 20%. In some embodiments, the method provided herein enhances wound healing by at least 30%. In some embodiments, the method provided herein enhances wound healing by at least 50%. In some embodiments, the method provided herein enhances wound healing by at least 70%. In some embodiments, the wound healing parameter is overall surface area of the wound, perimeter of the wound, S/P ratio of the wound, or margin distance from the wound center. In some embodiments, the wound healing parameter is overall surface area of the wound. In some embodiments, the preset degree is about 30%, 40%, 50%, 60%, 70%, 75%, 80%, 85%, 90%, 95% or 100% decrease. In some embodiments, the preset degree is 50%. In some embodiments, the preset degree is 95%. In some embodiments, the enhancement of wound healing is measured by the decease of time it takes to achieve 50% decrease of overall surface area of the wound. In some embodiments, the enhancement of wound healing is measured by the decease of time it takes to achieve 95% decrease of overall surface area of the wound. In some embodiments, the enhancement of wound healing is measured by the decease of time it takes to achieve full closure of the wound. In some embodiment, the wound is a chronic wound. In some embodiment, the wound is a diabetic wound.

In some embodiments, the method provided herein enhances wound healing in a subject, as compared to a control wound, by at least 5%, at least 10%, at least 15%, at least 20%, at least 25%, at least 30%, at least 40%, at least 50%, at least 60%, at least 70%, at least 80%, at least 90%, at least 1-fold, at least 2-fold, at least 3-fold, at least 4-fold, at least 5-fold, at least 7-fold, or at least 10-fold, including all ranges and subranges therebetween, as measured by the increase of the relative degree of the wound healing at a preset time point according to one of the wound healing parameters. In some embodiments, the method provided herein enhances wound healing by at least 10%. In some embodiments, the method provided herein enhances wound healing by at least 20%. In some embodiments, the method provided herein enhances wound healing by at least 30%. In some embodiments, the method provided herein enhances wound healing by at least 50%. In some embodiments, the method provided herein enhances wound healing by at least 70%. In some embodiments, the wound healing parameter is overall surface area of the wound, perimeter of the wound, S/P ratio of the wound, or margin distance from the wound center. In some embodiments, the wound healing parameter is overall surface area of the wound. In some embodiments, the preset time point is day 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 post wounding. In some embodiments, the preset time point is the end of week 1, week 2, week 3, week 4, week 5, week 6, week 7, week 8, week 9, week 10, week 11, week 12, week 13, week 14, week 15, week 16, week 17, week 18, week 19, or week 20 post wounding. In some embodiments, the preset time point is day 10 post wounding. In some embodiments, the preset time point is day 20 post wounding. In some embodiments, the preset time point is day 30 post wounding. In some embodiments, the enhancement of wound healing is measured by the decease of overall surface area of the wound at day 10 post wounding. In some embodiments, the enhancement of wound healing is measured by the decease of overall surface area of the wound at day 20 post wounding. In some embodiments, the enhancement of wound healing is measured by the decease of overall surface area of the wound at day 30 post wounding. In some embodiment, the wound is a chronic wound. In some embodiment, the wound is a diabetic wound.

In some embodiments the treatments of the present disclosure are effective at treating superficial wounds, such as those to the skin. In some embodiments, the treatments of the present disclosure at effective at treating internal wounds, such as wounds that may occur due to trauma or surgery.

In some embodiments the treatments are administered after the damage (e.g., wound) occurs. In some embodiments the treatments of the present disclosure are administered prophylactically, such as prior to embarking on an activity likely to cause a wound, or prior to a surgical procedure.

### EXAMPLES

### Example 1: Anti-Ceramide Antibody Accelerates Wound Healing in a Murine Model of Diabetes

To study the effect of anti-ceramide antibody on diabetic wound healing, a standardized punch biopsy-based murine surface wound model was used in a core facility at MSU.

In this study, mice were divided into four experimental groups:
(a) 1) Diabetic mice (6 animals) with application of anti-ceramide antibody 2A2 (Diabetic+2A2);
(b) 2) Diabetic mice (6 animals) without anti-ceramide antibody 2A2 (Diabetic);
(c) 3) Control mice (3 animals) with application of anti-ceramide antibody 2A2 (Control+2A2);
(d) 4) Control mice (5 animals) without anti-ceramide antibody 2A2 (Control).

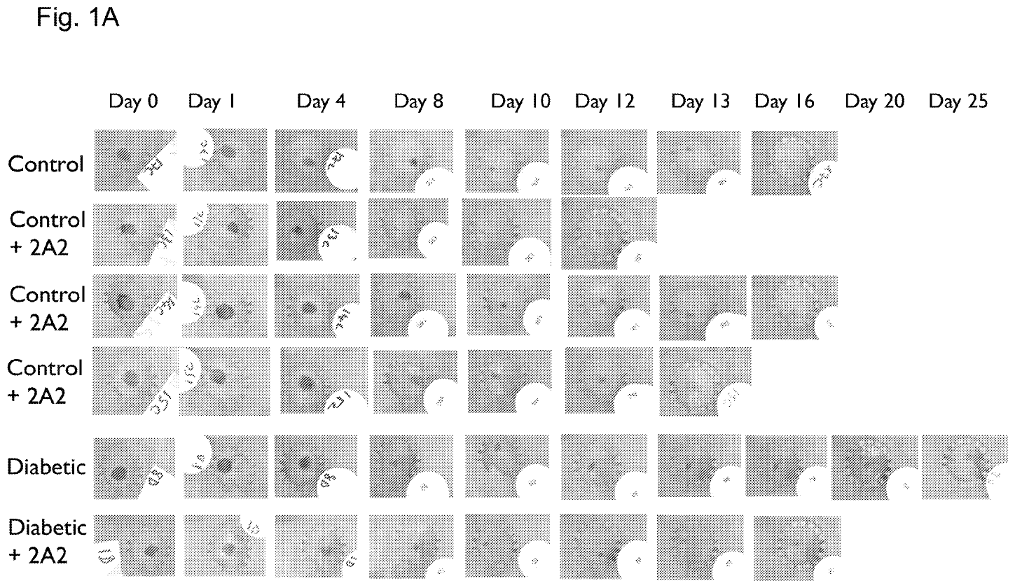

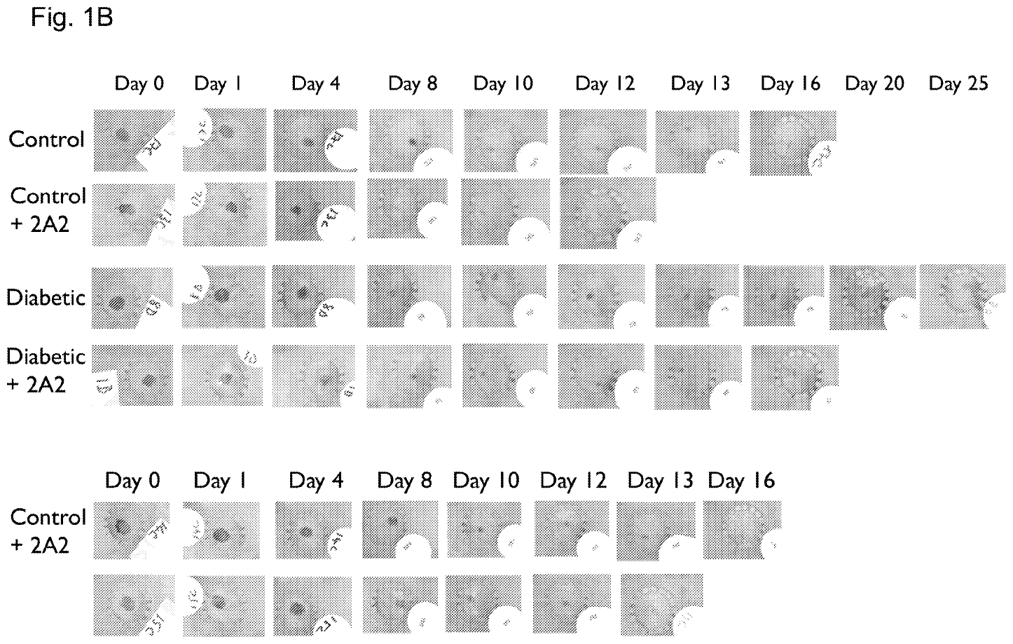

Wounds were introduced by 5 mm biopsy needle to the upper back of control (non-diabetic) and STZ-induced diabetic SKH1 hairless mice injected with either 1 mg of 2A2 anti-ceramide antibody or vehicle control (PBS) by IV injection every 4 days. Representative photos of wound site at days 0, 1, 4, 8, 10, 12, 13, 16, 20 and 25 post wounding are shown in FIG. 1A-FIG. 1B.

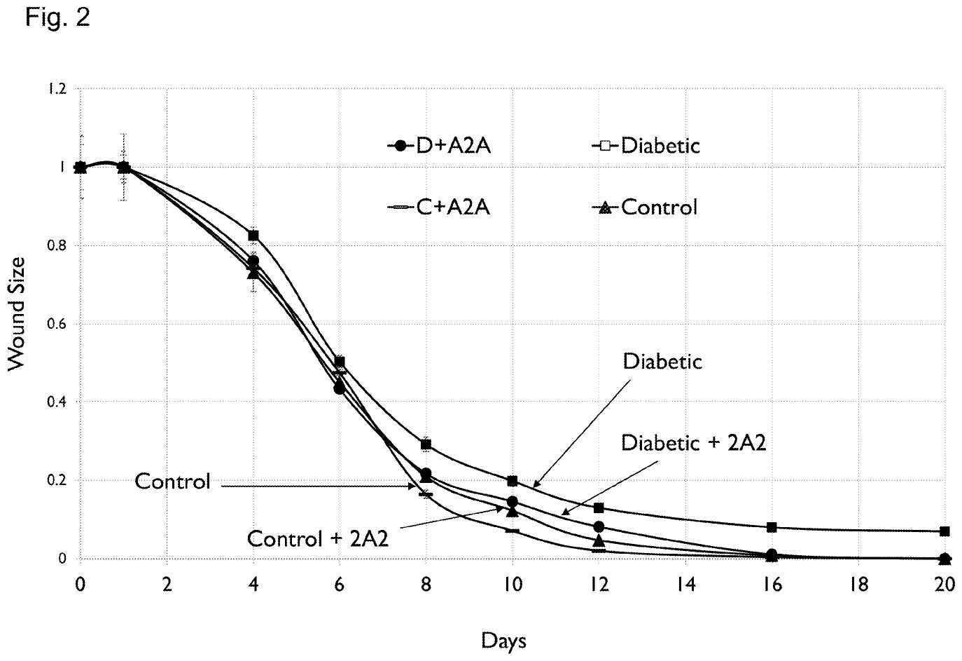

FIG. 2 shows the average rate of wound healing in each experimental group, expressed by the size of the lesion over time. For normalization of lesion size, the original size of the lesion in each case was set to 1. In both diabetic and non-diabetic mice, application of 2A2 antibody accelerated the wound healing process and this effect was more prominent in the diabetic mice group.

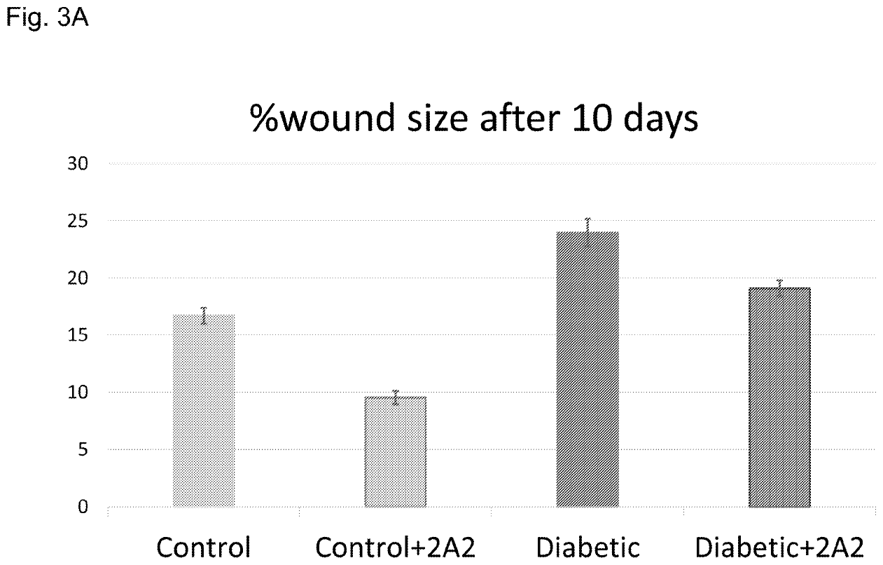

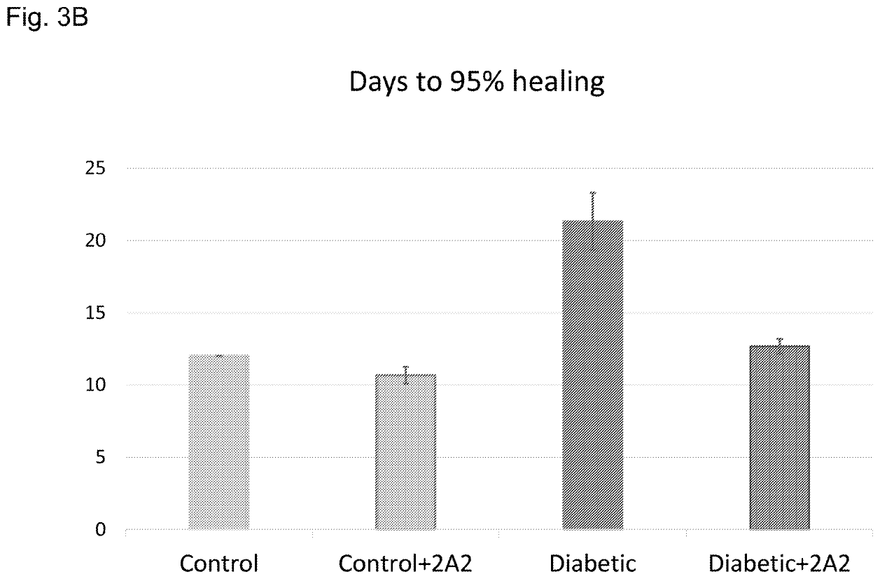

FIG. 3A shows the average wound closure calculated as % of the original wound size on Day 10. In both control group and diabetic group, a greater degree of wound closure was achieved after 10 days in mice injected with 2A2 than those injected with PBS vehicle control.

FIG. 3B shows the average number of days it took for the mice to achieve 95% wound closure (as compared to the original size of wound) in each experimental group. In the diabetic mice groups, application of 2A2 antibody significantly shortened the length of time it took to achieve 95% healing.

Overall, this study demonstrates that application of an anti-ceramide antibody significantly accelerates healing of wounds, and especially diabetic wounds.

### Study Protocol:

Below is the protocol of the animal study conducted in Example 1.

Study objective: to examine the effects of anti-ceramide antibody 2A2 on wound healing when administered intravenously (IV) once every 4 days to male SKH1 mice undergoing Streptozotocin (STZ) induced type I diabetes.

Male SKH1 mice, weighing between 20-25 g, were acclimated for approximately 2 weeks. Animals were divided to control (C) and diabetes induced (D) mice. Diabetes was made by intraperitoneal injections of streptozotocin (65 mg per kg of body weight) on five consecutive days. Two weeks after the last injection, blood glucose was measured from a drop of blood collected from the pedal dorsal vein and diabetes was confirmed by blood glucose levels >300 mg per dl. Weight loss, polyuria, water and food consumption were monitored daily and NPH insulin injections (0-2 units/day) was provided based on the clinical condition of the animal. Wounds were introduced as previously described after the diabetes is confirmed.

The animals were randomized to 2 groups for vehicle or 2A2 IV injection treatments.

Treatment started the day of wound introduction. IV dosing was continued every 4 days until complete healing of the wound (˜30 days). Control (C) animals were wounded on the same day as diabetic animals and randomized to 2 groups for vehicle or 2A2 IV injection treatments. The IV treatment was started the day of wounding. Dosing was continued every 4 days until complete healing of the wound (˜15 days). The IV dose of 2A2 antibody was 1 mg/25 g mouse every 4 days.

Wound measurements as pictures were taken right after the surgery and then every other day and healing time rates were calculated. At the end of the experiment wounds were excised, fixed and analyzed for dermal layer thickness, matrix deposition, re-vascularization, immune cell infiltration.

### Example 2: Further Experimental Validation

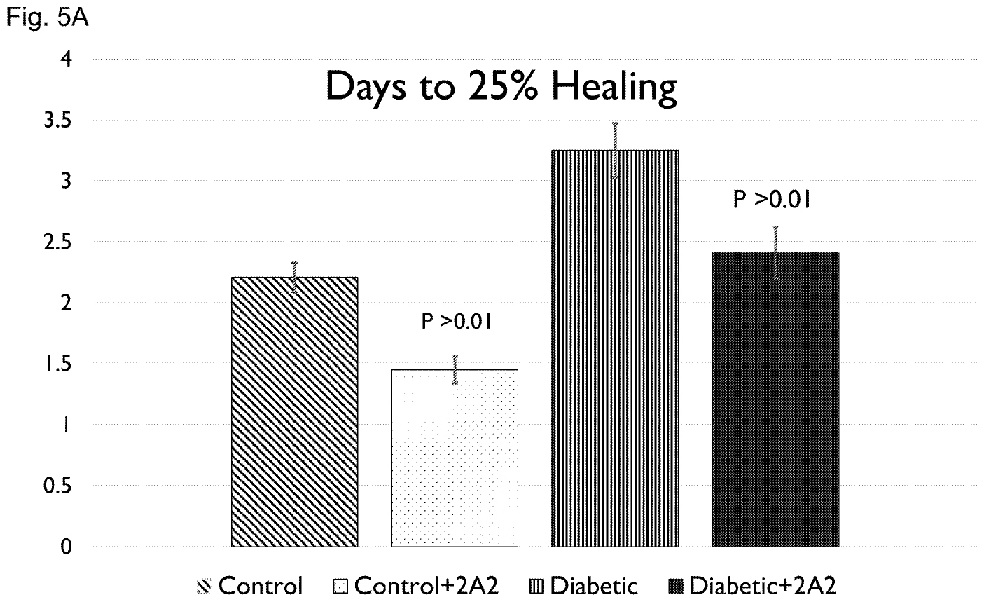

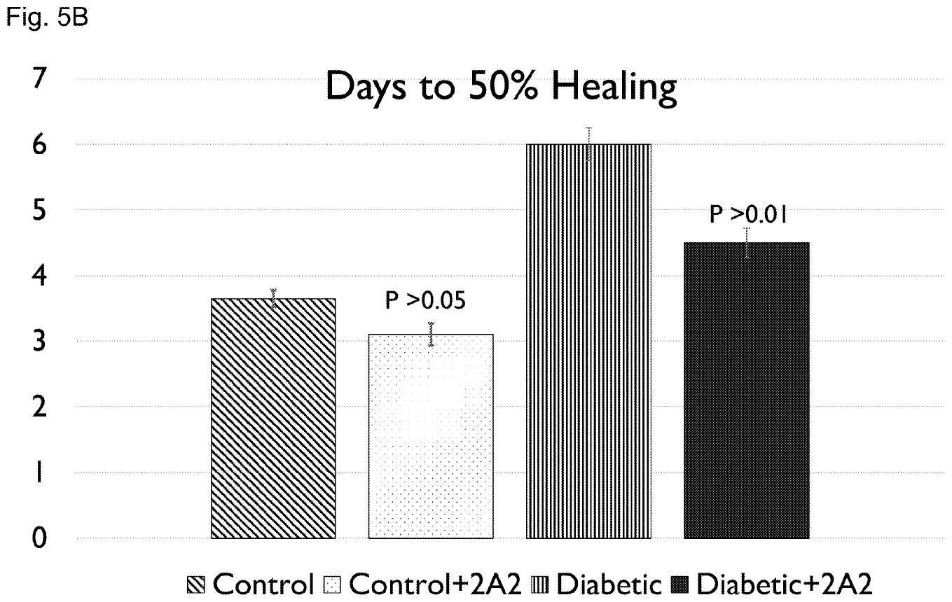

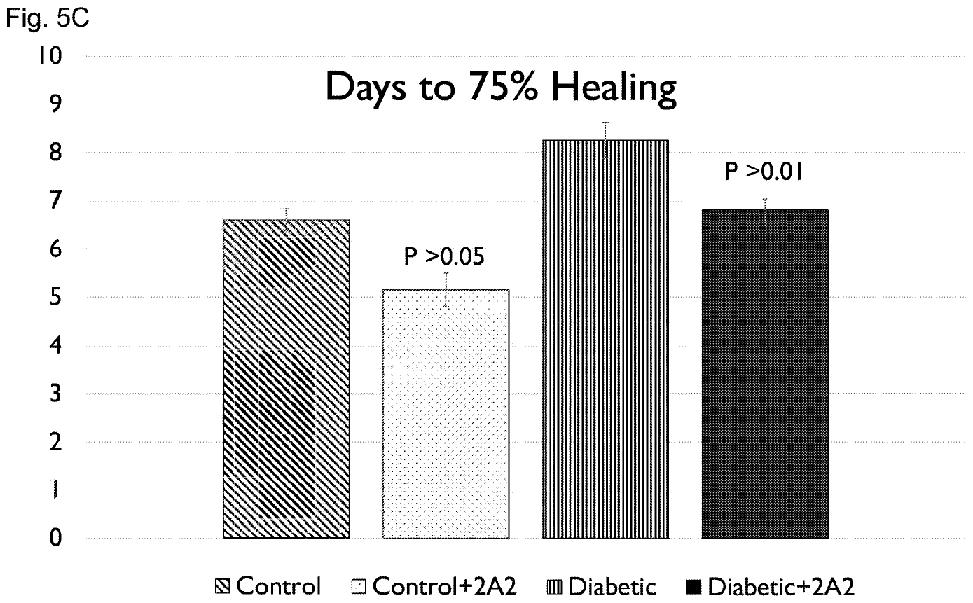

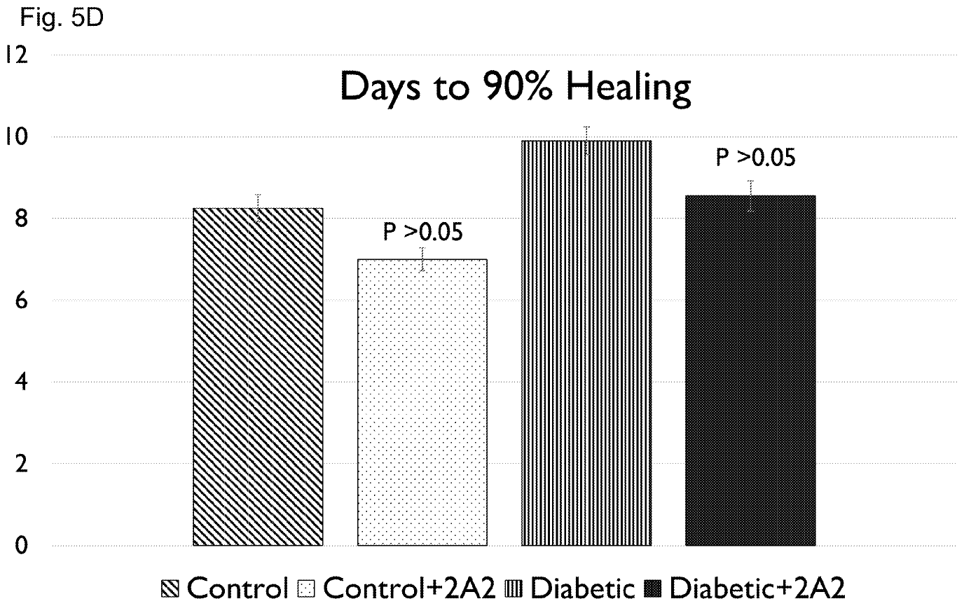

The experiments conducted in Example 1 were repeated with additional mice subjects. The results from this experiment are provided Tables 3-6 and FIGS. 3 and 5A-D.

TABLE 3
Results

| animal/day | 0 | 1 | 3 | 5 | 7 | 9 | 11 | 13 | 15 |
|---|---|---|---|---|---|---|---|---|---|
| 21C | 1 | 0.958736 | | 0.392653 | 0.345612 | 0.061131 | 0.012862 | 0 | |
| 22C | 1 | 0.86071 | 0.549365 | 0.35392 | 0.134691 | 0.01682 | 0 | | |
| 23C | 1 | 1.122077 | 0.545736 | 0.347662 | 0.193948 | 0.039202 | 0.007221 | 0 | |
| 24C | 1 | 1.082576 | | 0.407828 | 0.328535 | 0.118687 | 0.017677 | 0 | |
| 25C | 1 | 1.204009 | 0.66214 | 0.300679 | 0.155189 | 0.064662 | 0.04203 | 0.006466 | 0 |
| 26C | 1 | 0.870183 | 0.420642 | 0.357798 | 0.233945 | 0.018349 | 0 | | |
| 27C | 1 | 0.970573 | 0.498161 | | | 0.016658 | 0 | | |
| 28C | 1 | 1.035659 | 0.56157 | 0.370703 | | 0.038225 | 0.021242 | 0 | |
| 39C | 1 | 1.014643 | 0.752952 | 0.323571 | 0.161549 | 0 | | | |
| 40C | 1 | 0.945333 | 0.746489 | 0.408444 | 0.217333 | 0.044 | 0.013333 | 0 | |
| control | 1 | 1.00645 | 0.592132 | 0.362584 | 0.22135 | 0.041773 | 0.012707 | 0.001078 | 0 |
| stdev | 0 | 0.108639 | 0.118168 | 0.036734 | 0.078666 | 0.03397 | 0.013566 | 0.00264 | |
| sem | | 0.034355 | 0.037368 | 0.011616 | 0.024876 | 0.010742 | 0.00429 | 0.000835 | |

TABLE 4
Results

| animal/day | 0 | 1 | 3 | 5 | 7 | 9 | 11 | 13 | 15 |
|---|---|---|---|---|---|---|---|---|---|
| 29C | 1 | 0.729972 | 0.556079 | 0.222432 | | 0 | | | |
| 31C | 1 | 0.642781 | 0.484492 | 0.195009 | 0.164349 | 0 | | | |
| 32C | 1 | 0.785566 | 0.541018 | 0.288162 | 0.126687 | 0 | | | |
| 33C | 1 | 0.889613 | | | | | 0 | | |
| 34C | 1 | 0.876296 | 0.472593 | 0.327037 | 0.136667 | 0 | | | |
| 36C | 1 | 0.820911 | 0.519669 | 0.27588 | 0.087474 | 0 | | | |
| 37C | 1 | 0.810115 | | 0.212955 | 0.001 | 0 | | 0 | |
| 38C | 1 | 0.998926 | | 0.326531 | | 0 | | | |
| control + ab | 1 | 0.819272 | 0.51477 | 0.264001 | 0.103236 | 0 | 0 | 0 | |
| stdev | 0 | 0.107452 | 0.03576 | 0.054317 | 0.063449 | 0 | | | |
| sem | | 0.033979 | 0.011308 | 0.017177 | 0.020064 | 0 | 0 | 0 | |

TABLE 5
Results

| animal/day | 0 | 1 | 3 | 5 | 7 | 9 | 11 | 13 | 15 |
|---|---|---|---|---|---|---|---|---|---|
| 42D | 1 | 1.096244 | 0.845346 | 0.628487 | 0.574246 | 0.196345 | 0.068164 | 0.015648 | 0 |
| 43D | 1 | 1.029762 | 0.903161 | 0.46179 | 0.538839 | 0.204338 | 0.042348 | 0 | |
| 44D | 1 | 1.0369 | 0.848809 | 0.495202 | 0.434258 | 0 | | | |
| 45D | 1 | 1.014911 | 0.875051 | 0.443593 | 0.444703 | 0.097717 | 0.043808 | 0.013801 | 0 |
| 46D | 1 | 1.056953 | 0.735897 | 0.628315 | 0.376141 | 0.392023 | 0.114265 | 0 | |
| 47D | 1 | 1.023807 | 0.769287 | 0.669014 | 0.390504 | 0.085234 | 0 | | |
| 48D | 1 | 1.000784 | 0.526656 | 0.698706 | 0.416072 | 0.0608 | 0.023834 | 0 | |
| 49D | 1 | 1.000312 | 0.794277 | 0.660614 | 0.279605 | 0.405827 | 0.106139 | 0 | |
| 50D | 1 | 1.073402 | 0.721626 | 0.453354 | 0.3191 | 0 | | | |
| Diabetic | 1 | 1.037008 | 0.780012 | 0.571008 | 0.419274 | 0.160254 | 0.056937 | 0.004908 | 0 |
| stdev | 0 | 0.032797 | 0.113502 | 0.105048 | 0.094488 | 0.153322 | 0.041945 | 0.007626 | 0 |
| sem | | 0.010371 | 0.035893 | 0.033219 | 0.02988 | 0.048485 | 0.013264 | 0.002412 | 0 |

TABLE 6
Results

| animal/day | 0 | 1 | 3 | 5 | 7 | 9 | 11 | 13 | 15 |
|---|---|---|---|---|---|---|---|---|---|
| 51D | 1 | 1.067592 | 0.794211 | 0.507604 | 0.287121 | 0.039674 | 0.029608 | 0 | |
| 53D | 1 | 0.988004 | 0.873982 | 0.349428 | | 0.032594 | 0.22989 | 0.044895 | 0 |
| 54D | 1 | 1.06018 | 0.805195 | 0.540434 | 0.064242 | 0.03782 | 0.020893 | 0.030022 | 0.012715 |
| 55D | 1 | 1.139023 | 0.576737 | 0.489761 | 0.37781 | 0.085275 | 0.039659 | 0 | |
| 56D | 1 | 1.184604 | 0.537858 | 0.422259 | 0.190276 | 0.075715 | 0 | | |
| 57D | 1 | 1.199634 | 0.643747 | 0.583601 | 0.195419 | 0.019514 | 0 | | |
| 58D | 1 | 0.9585 | 0.436213 | 0.307409 | 0.209038 | 0.185951 | 0.050138 | 0 | |
| 59D | 1 | 0.868728 | 0.467256 | 0.333175 | 0.256518 | 0.093012 | 0 | | |
| 60D | 1 | 1.169255 | 0.57625 | 0.585949 | 0.251798 | 0.113 | 0.112101 | 0.030756 | 0 |
| Diabetic + ab | 1 | 1.070613 | 0.634605 | 0.457735 | 0.229028 | 0.075839 | 0.053588 | 0.017612 | 0.004238 |
| stdev | 0 | 0.114192 | 0.156301 | 0.108244 | 0.090214 | 0.052064 | 0.074976 | 0.020008 | 0.007341 |
| sem | | 0.036111 | 0.049427 | 0.03423 | 0.028528 | 0.016464 | 0.023709 | 0.006327 | 0.002321 |

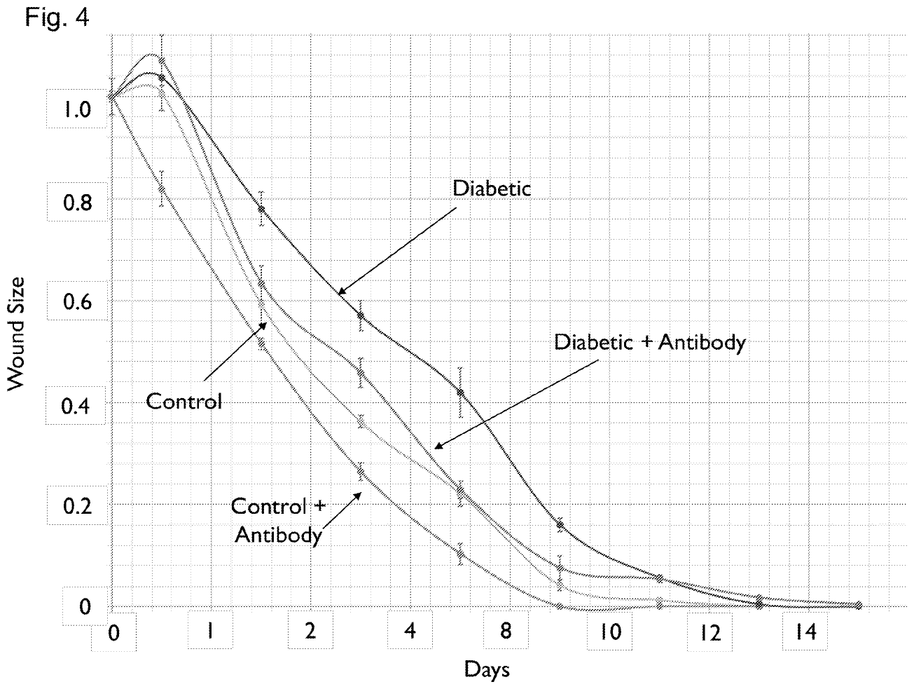

FIG. 4 shows the average rate of wound healing in each experimental group over time The Y axis indicates the size of the wound, with the starting size of each wound normalized as 1. The X axis indicates number of days.

FIG. 5A-D—are a bar charts showing the average number of days it took for the mice to achieve 25%, 50%, 75%, and 90% healing, respectively for each of the experimental groups.

This experiment further validates that the treatment of mice with anti-ceramide antibody in a statistically significant acceleration of wound healing, and especially diabetic wounds.

### Example 3: Topical Antibody Administration

The antibody treatment acceleration of wound healing described in Examples 1-2 are believed to be due to localized ceramide-signaling effects at the wound locus. It is therefore expected that topical administration of the presently disclosed anti-ceramide antibodies and/or antigen-binding fragments thereof will exhibit similar effects as described in Examples 1-2. The effect of these antibodies and antigen-binding fragments will be tested on Animal models, such as mice. The experiments will be conducted similar to the experiments described in Examples 1-2, except that the antibody will be administered topically, via a lotion, save, or other topical formulation known to those skilled in the art. In another experiment conducted similar to those described in Examples 1-2, the wounds will be treated via sub-cutaneous injections of the antibody or antigen-binding fragment thereof. Topical administrations of other ceramide signaling inhibitors, such as imipramine will be tested on wound healing according to methods of Example 1 and 2. It is expected that wounds treated with the antibody and/or antigen-binding fragments thereof (or imipramine) will accelerate/improve wound healing in control and/or diabetic animals.

### Example 4: Internal Wounds

Additional confirmatory experiments will be conducted to demonstrate the presently-disclosed treatments' ability to enhance/accelerate internal wounds. Experiments will be conducted on model animals in which an internal wound is inflicted on control and diabetic subjects and healing from the internal wound is tracked over time in subjects treated with vehicle/nothing, or with the anti-ceramide antibody or imipramine of the present disclosure. Healing will be tracked via any of the techniques known to those skilled in the art, including sonogram, MRI, CT, or visual assessment via surgical means.

### Example 5: Further Validations of Disclosed Antibodies and Antigen-Binding Fragments Thereof

This disclosure exemplifies the effects of anti-ceramide antibodies and/or antigen-binding fragments there of through validating experiments of the 2A2 antibody. The experiment of Examples 1-2 will be repeated using any one of the additional antibodies and antigen-binding fragments disclosed in this specification, or other antibodies/antigen-binding fragments capable of binding to ceramide or inhibiting the formation of ceramide-rich platforms. For instance, wound healing experiments as describes in Examples 1-2 will be repeated using the 6b5 murine (US 2019-0389970, which is hereby incorporated by reference), and 6b5 humanized (PCT/US2021/022914 and its corresponding U.S. provisional 62/991,232, both of which are hereby incorporated by reference), antibodies and fragments thereof.

### Further Numbered Embodiments

Further embodiments of the instant invention are provided in the numbered embodiments below:

Embodiment 1. A method of treating or preventing a wound in a subject in need thereof comprising administering an anti-ceramide antibody or antigen-binding fragment thereof to the subject.

Embodiment 2. A method of enhancing healing of a wound in a subject in need thereof, comprising administering an anti-ceramide antibody or antigen-binding fragment thereof to the subject.

Embodiment 3. The method of Embodiment 1 or 2, wherein the wound is a chronic wound or a diabetic wound.

Embodiment 4. The method of Embodiment 1 or 2, wherein the wound is a chronic wound.

Embodiment 5. The method of Embodiment 1 or 2, wherein the wound is a diabetic wound.

Embodiment 5.1 The method of Embodiment 1 or 2, wherein the wound an external wound.

Embodiment 5.2 The method of Embodiment 1 or 2, wherein the wound an internal wound.

Embodiment 5.3 The method of Embodiment 1 or 2, wherein the wound is on endothelial tissue.

Embodiment 6. The method of any one of Embodiments 1-5.3, wherein the route of administration is selected from the group consisting of topical administration, intralesional administration, subcutaneous administration, transdermal administration, intramuscular administration, intravenous administration, and parenteral administration.

Embodiment 7. The method of Embodiment 6, wherein the route of administration is topical administration.

Embodiment 8. The method of Embodiment 6, wherein the route of administration is intravenous administration.

Embodiment 9. The method of any one of Embodiments 1-8, wherein the anti-ceramide antibody or antigen-binding fragment thereof is administered as a single dose.

Embodiment 10. The method of any one of Embodiments 1-8, wherein the anti-ceramide antibody or antigen-binding fragment thereof is administered in two or more doses.

Embodiment 11. The method of Embodiment 10, wherein administrations of consecutive doses are separated by at least 1 day, at least 2 days, at least 3 days, at least 4 days, at least 5 days, or at least a week.

Embodiment 12. The method of Embodiment 10 or 11, wherein the duration of the administration is at least one week, at least two weeks, at least three weeks, or at least four weeks.

Embodiment 13. The method of any one of Embodiments 1-12, wherein the anti-ceramide antibody or antigen-binding fragment thereof is administered during the inflammatory stage of wound healing.

Embodiment 14. The method of any one of Embodiments 1-13, wherein the anti-ceramide antibody or antigen-binding fragment thereof is administered during the proliferative stage of wound healing.

Embodiment 15. The method of any one of Embodiments 1-14, wherein the anti-ceramide antibody or antigen-binding fragment thereof is administered during the remodeling stage of wound healing.

Embodiment 16. The method of any one of Embodiments 1-15, wherein the anti-ceramide antibody or antigen-binding fragment thereof is an antibody.

Embodiment 17. The method of any one of Embodiments 1-15, wherein the anti-ceramide antibody or antigen-binding fragment thereof is a single-chain variable fragment (scFv).

Embodiment 18. The method of any one of Embodiments 1-17, wherein the method enhances wound healing by at least 10%, at least 20%, at least 30%, at least 50%, or at least 70% as compared to a control wound.

Embodiment 19. The method of Embodiment 18, wherein the enhancement of wound healing is measured by the decease of overall surface area of the wound at day 10, 20 or 30 post wounding.

Embodiment 20. The method of Embodiment 19, wherein the enhancement of wound healing is measured at day 10 post wounding.

Embodiment 21. The method of Embodiment 18, wherein the enhancement of wound healing is measured by the decease of time it takes to achieve 50%, 70%, 90%, 95% or 100% decrease of overall surface area of the wound.

Embodiment 22. The method of Embodiments 21, wherein the enhancement of wound healing is measured at 95% decrease of overall surface area of the wound.

Embodiment 23. The method of any one of Embodiments 1-22, wherein the anti-ceramide antibody or antigen-binding fragment thereof is administered before the onset of one or more symptoms of the wound.

Embodiment 24. The method of any one of Embodiments 1-22, wherein the anti-ceramide antibody or antigen-binding fragment thereof is administered after the onset of one or more symptoms of the wound.

Embodiment 25. The method of any one of Embodiments 1-24, wherein the anti-ceramide antibody or antigen-binding fragment thereof comprises a variable heavy chain (VH) and a variable light chain (VL),
a) wherein the VH comprises a heavy chain complementarity determining region 1 (HCDR1) comprising the amino acid sequence of GYTFTDHTIH (SEQ ID NO: 1), an HCDR2 comprising the amino acid sequence of YNYPRDGSTKYNEKFKG (SEQ ID NO: 2), and an HCDR3 comprising the amino acid sequence of GFITTVVPSAY (SEQ ID NO: 3), and
b) wherein the VL comprises a light chain complementarity determining region 1 (LCDR1) comprising the amino acid sequence of RASKSISKYLA (SEQ ID NO: 4), an LCDR2 comprising the amino acid sequence of SGSTLQS (SEQ ID NO: 5), and an LCDR3 comprising the amino acid sequence of QQHNEYPWT (SEQ ID NO: 6).

Embodiment 26. The method of any one of Embodiments 1-25, wherein the VH comprises the amino acid sequence of SEQ ID NO: 7 and wherein the VL comprises the amino acid sequence of SEQ ID NO: 8.

Embodiment 27. The method of any one of Embodiments 1-26, wherein the anti-ceramide antibody or antigen-binding fragment thereof is a 6B5 antibody.

Embodiment 28. The method of any one of Embodiments 1-26, wherein the anti-ceramide antibody or antigen-binding fragment thereof is a 6B5 scFv.

Embodiment 29. The method of any one of Embodiments 1-24, wherein the anti-ceramide antibody or antigen-binding fragment thereof comprises a variable heavy chain (VH) and a variable light chain (VL),
a) wherein the VH comprises a heavy chain complementarity determining region 1 (HCDR1) comprising the amino acid sequence of NYWMH (SEQ ID NO: 33), an HCDR2 comprising the amino acid sequence of AIYPGDSDTSYNQKFKG (SEQ ID NO: 34), and an HCDR3 comprising the amino acid sequence of LYYGYD (SEQ ID NO: 35), and
b) wherein the VL comprises a light chain complementarity determining region 1 (LCDR1) comprising the amino acid sequence of KSSQSLIDSDGKTFLN (SEQ ID NO: 36), an LCDR2 comprising the amino acid sequence of LVSKLDS (SEQ ID NO: 37), and an LCDR3 comprising the amino acid sequence of WQGTHFPYT (SEQ ID NO: 38).

Embodiment 30. The method of any one of Embodiments 1-29, wherein the VH comprises the amino acid sequence of SEQ ID NO: 39 and wherein the VL comprises the amino acid sequence of SEQ ID NO: 40.

Embodiment 31. The method of any one of Embodiments 1-24 and 29-30, wherein the anti-ceramide antibody or antigen-binding fragment thereof is a 2A2 antibody.

Embodiment 32. The method of any one of Embodiments 1-24 and 29-30, wherein the anti-ceramide antibody or antigen-binding fragment thereof is a 2A2 scFv.

Embodiment 33. The method of any one of Embodiments 1-24, wherein the anti-ceramide antibody or antigen-binding fragment thereof comprises a variable heavy chain (VH) and a variable light chain (VL),
a) wherein the VH comprises a heavy chain complementarity determining region 1 (HCDR1) comprising or consisting of an amino acid sequence selected from the group consisting of SEQ ID NOS: 1 and 43; an HCDR2 comprising or consisting of an amino acid sequence selected from the group consisting of SEQ ID NOS: 44-47, and an HCDR3 comprising or consisting of an amino acid sequence of GFITTVVPSAY (SEQ ID NO: 3), and
b) wherein the VL comprises a light chain complementarity determining region 1 (LCDR1) comprising or consisting of an amino acid sequence of RASKSISKYLA (SEQ ID NO: 4), an LCDR2 comprising or consisting of an amino acid sequence of SGSTLQS (SEQ ID NO: 5), and an LCDR3 comprising or consisting of an amino acid sequence of QQHNEYPWT (SEQ ID NO: 6).

Embodiment 34. The method of Embodiment 33, wherein the HCDR1 comprises or consists of the amino acid sequence of GYTFTDHTIH (SEQ ID NO: 1), and the HCDR2 comprises or consists of the amino acid sequence of YNYPRDGSTKYNEKFQG (SEQ ID NO: 44).

Embodiment 35. The method of Embodiment 33, wherein the HCDR1 comprises or consists of the amino acid sequence of GYTFTDHTIH (SEQ ID NO: 1), and the HCDR2 comprises or consists of the amino acid sequence of YNYPREGSTKYNEKFQG (SEQ ID NO: 45).

Embodiment 36. The method of Embodiment 33, wherein the HCDR1 comprises or consists of the amino acid sequence of GYTFTDHTIH (SEQ ID NO: 1), and the HCDR2 comprises or consists of the amino acid sequence of YNYPRDVSTKYNEKFQG (SEQ ID NO: 46).

Embodiment 37. The method of Embodiment 33, wherein the HCDR1 comprises or consists of the amino acid sequence of GYTFTDHTIH (SEQ ID NO: 1), and the HCDR2 comprises or consists of the amino acid sequence of YNYPRDGSTKYAEKFQG (SEQ ID NO: 47).

Embodiment 38. The method of Embodiment 33, wherein the HCDR1 comprises or consists of the amino acid sequence of GYTFTDHTMH (SEQ ID NO: 43), and the HCDR2 comprises or consists of the amino acid sequence of YNYPRDGSTKYNEKFQG (SEQ ID NO: 44).

Embodiment 39. The method of Embodiment 33, wherein the HCDR1 comprises or consists of the amino acid sequence of GYTFTDHTMH (SEQ ID NO: 43), and the HCDR2 comprises or consists of the amino acid sequence of YNYPREGSTKYNEKFQG (SEQ ID NO: 45).

Embodiment 40. The method of Embodiment 33, wherein the HCDR1 comprises or consists of the amino acid sequence of GYTFTDHTMH (SEQ ID NO: 43), and the HCDR2 comprises or consists of the amino acid sequence of YNYPRDVSTKYNEKFQG (SEQ ID NO: 46).

Embodiment 41. The method of Embodiment 33, wherein the HCDR1 comprises or consists of the amino acid sequence of GYTFTDHTMH (SEQ ID NO: 43), and the HCDR2 comprises or consists of the amino acid sequence of YNYPRDGSTKYAEKFQG (SEQ ID NO: 47).

Embodiment 42. The method of Embodiment 33, wherein the VH comprises or consists of an amino acid sequence that is at least 90%, at least 95%, at least 97% identical, or 100% identical to SEQ ID NO: 48 and the VL comprises or consists of an amino acid sequence that is at least 90%, at least 95%, at least 97% identical, or 100% identical to SEQ ID NO: 53.

Embodiment 43. The method of Embodiment 33, wherein the VH comprises or consists of an amino acid sequence that is at least 90%, at least 95%, at least 97% identical, or 100% identical to SEQ ID NO: 48 and the VL comprises or consists of an amino acid sequence that is at least 90%, at least 95%, at least 97% identical, or 100% identical to SEQ ID NO: 54.

Embodiment 44. The method of Embodiment 33, wherein the VH comprises or consists of an amino acid sequence that is at least 90%, at least 95%, at least 97% identical, or 100% identical to SEQ ID NO: 48 and the VL comprises or consists of an amino acid sequence that is at least 90%, at least 95%, at least 97% identical, or 100% identical to SEQ ID NO: 55.

Embodiment 45. The method of Embodiment 33, wherein the VH comprises or consists of an amino acid sequence that is at least 90%, at least 95%, at least 97% identical, or 100% identical to SEQ ID NO: 49 and the VL comprises or consists of an amino acid sequence that is at least 90%, at least 95%, at least 97% identical, or 100% identical to SEQ ID NO: 53.

Embodiment 46. The method of Embodiment 33, wherein the VH comprises or consists of an amino acid sequence that is at least 90%, at least 95%, at least 97% identical, or 100% identical to SEQ ID NO: 49 and the VL comprises or consists of an amino acid sequence that is at least 90%, at least 95%, at least 97% identical, or 100% identical to SEQ ID NO: 54.

Embodiment 47. The method of Embodiment 33, wherein the VH comprises or consists of an amino acid sequence that is at least 90%, at least 95%, at least 97% identical, or 100% identical to SEQ ID NO: 49 and the VL comprises or consists of an amino acid sequence that is at least 90%, at least 95%, at least 97% identical, or 100% identical to SEQ ID NO: 55.

Embodiment 48. The method of Embodiment 33, wherein the VH comprises or consists of an amino acid sequence that is at least 90%, at least 95%, at least 97% identical, or 100% identical to SEQ ID NO: 50 and the VL comprises or consists of an amino acid sequence that is at least 90%, at least 95%, at least 97% identical, or 100% identical to SEQ ID NO: 53.

Embodiment 49. The method of Embodiment 33, wherein the VH comprises or consists of an amino acid sequence that is at least 90%, at least 95%, at least 97% identical, or 100% identical to SEQ ID NO: 50 and the VL comprises or consists of an amino acid sequence that is at least 90%, at least 95%, at least 97% identical, or 100% identical to SEQ ID NO: 54.

Embodiment 50. The method of Embodiment 33, wherein the VH comprises or consists of an amino acid sequence that is at least 90%, at least 95%, at least 97% identical, or 100% identical to SEQ ID NO: 50 and the VL comprises or consists of an amino acid sequence that is at least 90%, at least 95%, at least 97% identical, or 100% identical to SEQ ID NO: 55.

Embodiment 51. The method of Embodiment 33, wherein the VH comprises or consists of an amino acid sequence that is at least 90%, at least 95%, at least 97% identical, or 100% identical to SEQ ID NO: 51 and the VL comprises or consists of an amino acid sequence that is at least 90%, at least 95%, at least 97% identical, or 100% identical to SEQ ID NO: 53.

Embodiment 52. The method of Embodiment 33, wherein the VH comprises or consists of an amino acid sequence that is at least 90%, at least 95%, at least 97% identical, or 100% identical to SEQ ID NO: 51 and the VL comprises or consists of an amino acid sequence that is at least 90%, at least 95%, at least 97% identical, or 100% identical to SEQ ID NO: 54.

Embodiment 53. The method of Embodiment 33, wherein the VH comprises or consists of an amino acid sequence that is at least 90%, at least 95%, at least 97% identical, or 100% identical to SEQ ID NO: 51 and the VL comprises or consists of an amino acid sequence that is at least 90%, at least 95%, at least 97% identical, or 100% identical to SEQ ID NO: 55.

Embodiment 54. The method of Embodiment 33, wherein the VH comprises or consists of an amino acid sequence that is at least 90%, at least 95%, at least 97% identical, or 100% identical to SEQ ID NO: 52 and the VL comprises or consists of an amino acid sequence that is at least 90%, at least 95%, at least 97% identical, or 100% identical to SEQ ID NO: 53.

Embodiment 55. The method of Embodiment 33, wherein the VH comprises or consists of an amino acid sequence that is at least 90%, at least 95%, at least 97% identical, or 100% identical to SEQ ID NO: 52 and the VL comprises or consists of an amino acid sequence that is at least 90%, at least 95%, at least 97% identical, or 100% identical to SEQ ID NO: 54.

Embodiment 56. The method of Embodiment 33, wherein the VH comprises or consists of an amino acid sequence that is at least 90%, at least 95%, at least 97% identical, or 100% identical to SEQ ID NO: 52 and the VL comprises or consists of an amino acid sequence that is at least 90%, at least 95%, at least 97% identical, or 100% identical to SEQ ID NO: 55.

Embodiment 57. The method of any one of Embodiments 33-56, wherein the anti-ceramide antibody or antigen-binding fragment thereof is a humanized 6B5 (h6B5) antibody.

Embodiment 58. The method of any one of Embodiments 33-56, wherein the anti-ceramide antibody or antigen-binding fragment thereof is a h6B5 scFv.

Embodiment 59. The method of any one of Embodiments 1-24, wherein the anti-ceramide antibody or antigen-binding fragment thereof comprises a variable heavy chain (VH) and a variable light chain (VL), wherein the VH comprises an amino acid sequence of SEQ ID NO: 48, and wherein the VL comprises an amino acid sequence of SEQ ID NO: 53.

Embodiment 60. The method of any one of Embodiments 1-24, wherein the anti-ceramide antibody or antigen-binding fragment thereof comprises a variable heavy chain (VH) and a variable light chain (VL), wherein the VH comprises an amino acid sequence of SEQ ID NO: 48, and wherein the VL comprises an amino acid sequence of SEQ ID NO: 55.

Embodiment 61. The method of any one of Embodiments 1-24, wherein the anti-ceramide antibody or antigen-binding fragment thereof comprises a variable heavy chain (VH) and a variable light chain (VL), wherein the VH comprises an amino acid sequence of SEQ ID NO: 49, and wherein the VL comprises an amino acid sequence of SEQ ID NO: 53.

Embodiment 62. The method of any one of Embodiments 1-24, wherein the anti-ceramide antibody or antigen-binding fragment thereof comprises a variable heavy chain (VH) and a variable light chain (VL), wherein the VH comprises an amino acid sequence of SEQ ID NO: 49, and wherein the VL comprises an amino acid sequence of SEQ ID NO: 54.

Embodiment 63. The method of any one of Embodiments 1-24, wherein the anti-ceramide antibody or antigen-binding fragment thereof comprises a variable heavy chain (VH) and a variable light chain (VL), wherein the VH comprises an amino acid sequence of SEQ ID NO: 50, and wherein the VL comprises an amino acid sequence of SEQ ID NO: 53.

Embodiment 64. The method of any one of Embodiments 1-24, wherein the anti-ceramide antibody or antigen-binding fragment thereof comprises a variable heavy chain (VH) and a variable light chain (VL), wherein the VH comprises an amino acid sequence of SEQ ID NO: 50, and wherein the VL comprises an amino acid sequence of SEQ ID NO: 54.

Embodiment 65. The method of any one of Embodiments 1-24, wherein the anti-ceramide antibody or antigen-binding fragment thereof comprises a variable heavy chain (VH) and a variable light chain (VL), wherein the VH comprises an amino acid sequence of SEQ ID NO: 51, and wherein the VL comprises an amino acid sequence of SEQ ID NO: 53.

Embodiment 66. The method of any one of Embodiments 1-24, wherein the anti-ceramide antibody or antigen-binding fragment thereof comprises a variable heavy chain (VH) and a variable light chain (VL), wherein the VH comprises an amino acid sequence of SEQ ID NO: 52, and wherein the VL comprises an amino acid sequence of SEQ ID NO: 53.

Embodiment 67. The method of any one of Embodiments 59-66, wherein the anti-ceramide antibody or antigen-binding fragment thereof is a humanized antibody.

Embodiment 68. The method of any one of Embodiments 59-66, wherein the anti-ceramide antibody or antigen-binding fragment thereof is a humanized scFv.

Embodiment 69. A method of treating or preventing a wound in a subject in need thereof comprising administering imipramine or a salt thereof to the subject.

Embodiment 70. A method of enhancing healing of a wound in a subject in need thereof, comprising administering imipramine or a salt thereof to the subject.

Embodiment 71. The method of Embodiment 70 or 71, wherein the wound is a chronic wound or a diabetic wound.

Embodiment 72. The method of Embodiment 70 or 71, wherein the wound is a chronic wound.

Embodiment 73. The method of Embodiment 70 or 712, wherein the wound is a diabetic wound.

Embodiment 74. The method of any one of Embodiments 70-73, wherein the route of administration is selected from the group consisting of topical administration, intralesional administration, subcutaneous administration, transdermal administration, intramuscular administration, intravenous administration, and parenteral administration.

Embodiment 75. The method of Embodiment 74, wherein the route of administration is topical administration.

Embodiment 76. The method of Embodiment 74, wherein the route of administration is intravenous administration.

Embodiment 77. The method of any one of Embodiments 70-76, wherein the anti-ceramide antibody or antigen-binding fragment thereof is administered as a single dose.

Embodiment 78. The method of any one of Embodiments 1-8, wherein the anti-ceramide antibody or antigen-binding fragment thereof is administered in two or more doses.

Embodiment 79. The method of Embodiment 78, wherein administrations of consecutive doses are separated by at least 1 day, at least 2 days, at least 3 days, at least 4 days, at least 5 days, or at least a week.

Embodiment 80. The method of Embodiment 78 or 79, wherein the duration of the administration is at least one week, at least two weeks, at least three weeks, or at least four weeks.

Embodiment 81. The method of any one of Embodiments 70-80, wherein the anti-ceramide antibody or antigen-binding fragment thereof is administered during the inflammatory stage of wound healing.

Embodiment 82. The method of any one of Embodiments 70-81, wherein the anti-ceramide antibody or antigen-binding fragment thereof is administered during the proliferative stage of wound healing.

Embodiment 83. The method of any one of Embodiments 70-82, wherein the anti-ceramide antibody or antigen-binding fragment thereof is administered during the remodeling stage of wound healing.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

1. A method of treating or preventing a wound in a subject in need thereof comprising administering an anti-ceramide antibody or antigen-binding fragment thereof to the subject.

2. A method of enhancing healing of a wound in a subject in need thereof, comprising administering an anti-ceramide antibody or antigen-binding fragment thereof to the subject.

3. The method of claim 1 or 2, wherein the wound is a chronic wound or a diabetic wound.

4. The method of claim 1 or 2, wherein the wound is a chronic wound.

5. The method of claim 1 or 2, wherein the wound is a diabetic wound.

6. The method of any one of claims 1-5, wherein the route of administration is selected from the group consisting of topical administration, intralesional administration, subcutaneous administration, transdermal administration, intramuscular administration, intravenous administration, and parenteral administration.

7. The method of claim 6, wherein the route of administration is topical administration.

8. The method of claim 6, wherein the route of administration is intravenous administration.

9. The method of any one of claims 1-8, wherein the anti-ceramide antibody or antigen-binding fragment thereof is administered as a single dose.

10. The method of any one of claims 1-8, wherein the anti-ceramide antibody or antigen-binding fragment thereof is administered in two or more doses.

11. The method of claim 10, wherein administrations of consecutive doses are separated by at least 1 day, at least 2 days, at least 3 days, at least 4 days, at least 5 days, or at least a week.

12. The method of claim 10 or 11, wherein the duration of the administration is at least one week, at least two weeks, at least three weeks, or at least four weeks.

13. The method of any one of claims 1-12, wherein the anti-ceramide antibody or antigen-binding fragment thereof is administered during the inflammatory stage of wound healing.

14. The method of any one of claims 1-13, wherein the anti-ceramide antibody or antigen-binding fragment thereof is administered during the proliferative stage of wound healing.

15. The method of any one of claims 1-14, wherein the anti-ceramide antibody or antigen-binding fragment thereof is administered during the remodeling stage of wound healing.

16. The method of any one of claims 1-15, wherein the anti-ceramide antibody or antigen-binding fragment thereof is an antibody.

17. The method of any one of claims 1-15, wherein the anti-ceramide antibody or antigen-binding fragment thereof is a single-chain variable fragment (scFv).

18. The method of any one of claims 1-17, wherein the method enhances wound healing by at least 10%, at least 20%, at least 30%, at least 50%, or at least 70% as compared to a control wound.

19. The method of claim 18, wherein the enhancement of wound healing is measured by the decease of overall surface area of the wound at day 10, 20 or 30 post wounding.

20. The method of claim 19, wherein the enhancement of wound healing is measured at day 10 post wounding.

21. The method of claim 18, wherein the enhancement of wound healing is measured by the decease of time it takes to achieve 50%, 70%, 90%, 95% or 100% decrease of overall surface area of the wound.

22. The method of claim 21, wherein the enhancement of wound healing is measured at 95% decrease of overall surface area of the wound.

23. The method of any one of claims 1-22, wherein the anti-ceramide antibody or antigen-binding fragment thereof is administered before the onset of one or more symptoms of the wound.

24. The method of any one of claims 1-22, wherein the anti-ceramide antibody or antigen-binding fragment thereof is administered after the onset of one or more symptoms of the wound.

25. The method of any one of claims 1-24, wherein the anti-ceramide antibody or antigen-binding fragment thereof comprises a variable heavy chain (V_{H}) and a variable light chain (V_{L}),
a) wherein the VH comprises a heavy chain complementarity determining region 1 (HCDR1) comprising the amino acid sequence of GYTFTDHTIH (SEQ ID NO: 1), an HCDR2 comprising the amino acid sequence of YNYPRDGSTKYNEKFKG (SEQ ID NO: 2), and an HCDR3 comprising the amino acid sequence of GFITTVVPSAY (SEQ ID NO: 3), and
b) wherein the VL comprises a light chain complementarity determining region 1 (LCDR1) comprising the amino acid sequence of RASKSISKYLA (SEQ ID NO: 4), an LCDR2 comprising the amino acid sequence of SGSTLQS (SEQ ID NO: 5), and an LCDR3 comprising the amino acid sequence of QQHNEYPWT (SEQ ID NO: 6).

26. The method of any one of claims 1-25, wherein the V_{H} comprises the amino acid sequence of SEQ ID NO: 7 and wherein the V_{L} comprises the amino acid sequence of SEQ ID NO: 8.

27. The method of any one of claims 1-26, wherein the anti-ceramide antibody or antigen-binding fragment thereof is a 6B5 antibody.

28. The method of any one of claims 1-26, wherein the anti-ceramide antibody or antigen-binding fragment thereof is a 6B5 scFv.

29. The method of any one of claims 1-24, wherein the anti-ceramide antibody or antigen-binding fragment thereof comprises a variable heavy chain (V_{H}) and a variable light chain (V_{L}),
a) wherein the V_{H} comprises a heavy chain complementarity determining region 1 (HCDR1) comprising the amino acid sequence of NYWMH (SEQ ID NO: 33), an HCDR2 comprising the amino acid sequence of AIYPGDSDTSYNQKFKG (SEQ ID NO: 34), and an HCDR3 comprising the amino acid sequence of LYYGYD (SEQ ID NO: 35), and
b) wherein the V_{L} comprises a light chain complementarity determining region 1 (LCDR1) comprising the amino acid sequence of KSSQSLIDSDGKTFLN (SEQ ID NO: 36), an LCDR2 comprising the amino acid sequence of LVSKLDS (SEQ ID NO: 37), and an LCDR3 comprising the amino acid sequence of WQGTHFPYT (SEQ ID NO: 38).

30. The method of any one of claims 1-29, wherein the V_{H} comprises the amino acid sequence of SEQ ID NO: 39 and wherein the V_{L} comprises the amino acid sequence of SEQ ID NO: 40.

31. The method of any one of claims 1-24 and 29-30, wherein the anti-ceramide antibody or antigen-binding fragment thereof is a 2A2 antibody.

32. The method of any one of claims 1-24 and 29-30, wherein the anti-ceramide antibody or antigen-binding fragment thereof is a 2A2 scFv.

33. The method of any one of claims 1-24, wherein the anti-ceramide antibody or antigen-binding fragment thereof comprises a variable heavy chain (V_{H}) and a variable light chain (V_{L}),
a) wherein the V_{H} comprises a heavy chain complementarity determining region 1 (HCDR1) comprising or consisting of an amino acid sequence selected from SEQ ID NOS: 1 and 43, an HCDR2 comprising or consisting of an amino acid sequence selected from SEQ ID NOS: 44-47, and an HCDR3 comprising or consisting of an amino acid sequence of GFITTVVPSAY (SEQ ID NO: 3), and
b) wherein the V_{L} comprises a light chain complementarity determining region 1 (LCDR1) comprising or consisting of an amino acid sequence of RASKSISKYLA (SEQ ID NO: 4), an LCDR2 comprising or consisting of an amino acid sequence of SGSTLQS (SEQ ID NO: 5), and an LCDR3 comprising or consisting of an amino acid sequence of QQHNEYPWT (SEQ ID NO: 6).

34. The method of claim 33, wherein the HCDR1 comprises or consists of the amino acid sequence of GYTFTDHTIH (SEQ ID NO: 1), and the HCDR2 comprises or consists of the amino acid sequence of YNYPRDGSTKYNEKFQG (SEQ ID NO: 44).

35. The method of claim 33, wherein the HCDR1 comprises or consists of the amino acid sequence of GYTFTDHTIH (SEQ ID NO: 1), and the HCDR2 comprises or consists of the amino acid sequence of YNYPREGSTKYNEKFQG (SEQ ID NO: 45).

36. The method of claim 33, wherein the HCDR1 comprises or consists of the amino acid sequence of GYTFTDHTIH (SEQ ID NO: 1), and the HCDR2 comprises or consists of the amino acid sequence of YNYPRDVSTKYNEKFQG (SEQ ID NO: 46).

37. The method of claim 33, wherein the HCDR1 comprises or consists of the amino acid sequence of GYTFTDHTIH (SEQ ID NO: 1), and the HCDR2 comprises or consists of the amino acid sequence of YNYPRDGSTKYAEKFQG (SEQ ID NO: 47).

38. The method of claim 33, wherein the HCDR1 comprises or consists of the amino acid sequence of GYTFTDHTMH (SEQ ID NO: 43), and the HCDR2 comprises or consists of the amino acid sequence of YNYPRDGSTKYNEKFQG (SEQ ID NO: 44).

39. The method of claim 33, wherein the HCDR1 comprises or consists of the amino acid sequence of GYTFTDHTMH (SEQ ID NO: 43), and the HCDR2 comprises or consists of the amino acid sequence of YNYPREGSTKYNEKFQG (SEQ ID NO: 45).

40. The method of claim 33, wherein the HCDR1 comprises or consists of the amino acid sequence of GYTFTDHTMH (SEQ ID NO: 43), and the HCDR2 comprises or consists of the amino acid sequence of YNYPRDVSTKYNEKFQG (SEQ ID NO: 46).

41. The method of claim 33, wherein the HCDR1 comprises or consists of the amino acid sequence of GYTFTDHTMH (SEQ ID NO: 43), and the HCDR2 comprises or consists of the amino acid sequence of YNYPRDGSTKYAEKFQG (SEQ ID NO: 47).

42. The method of claim 33, wherein the VH comprises or consists of an amino acid sequence that is at least 90%, at least 95%, at least 97% identical, or 100% identical to SEQ ID NO: 48 and the VL comprises or consists of an amino acid sequence that is at least 90%, at least 95%, at least 97% identical, or 100% identical to SEQ ID NO: 53.

43. The method of claim 33, wherein the VH comprises or consists of an amino acid sequence that is at least 90%, at least 95%, at least 97% identical, or 100% identical to SEQ ID NO: 48 and the VL comprises or consists of an amino acid sequence that is at least 90%, at least 95%, at least 97% identical, or 100% identical to SEQ ID NO: 54.

44. The method of claim 33, wherein the VH comprises or consists of an amino acid sequence that is at least 90%, at least 95%, at least 97% identical, or 100% identical to SEQ ID NO: 48 and the VL comprises or consists of an amino acid sequence that is at least 90%, at least 95%, at least 97% identical, or 100% identical to SEQ ID NO: 55.

45. The method of claim 33, wherein the VH comprises or consists of an amino acid sequence that is at least 90%, at least 95%, at least 97% identical, or 100% identical to SEQ ID NO: 49 and the VL comprises or consists of an amino acid sequence that is at least 90%, at least 95%, at least 97% identical, or 100% identical to SEQ ID NO: 53.

46. The method of claim 33, wherein the VH comprises or consists of an amino acid sequence that is at least 90%, at least 95%, at least 97% identical, or 100% identical to SEQ ID NO: 49 and the VL comprises or consists of an amino acid sequence that is at least 90%, at least 95%, at least 97% identical, or 100% identical to SEQ ID NO: 54.

47. The method of claim 33, wherein the VH comprises or consists of an amino acid sequence that is at least 90%, at least 95%, at least 97% identical, or 100% identical to SEQ ID NO: 49 and the VL comprises or consists of an amino acid sequence that is at least 90%, at least 95%, at least 97% identical, or 100% identical to SEQ ID NO: 55.

48. The method of claim 33, wherein the VH comprises or consists of an amino acid sequence that is at least 90%, at least 95%, at least 97% identical, or 100% identical to SEQ ID NO: 50 and the VL comprises or consists of an amino acid sequence that is at least 90%, at least 95%, at least 97% identical, or 100% identical to SEQ ID NO: 53.

49. The method of claim 33, wherein the VH comprises or consists of an amino acid sequence that is at least 90%, at least 95%, at least 97% identical, or 100% identical to SEQ ID NO: 50 and the VL comprises or consists of an amino acid sequence that is at least 90%, at least 95%, at least 97% identical, or 100% identical to SEQ ID NO: 54.

50. The method of claim 33, wherein the VH comprises or consists of an amino acid sequence that is at least 90%, at least 95%, at least 97% identical, or 100% identical to SEQ ID NO: 50 and the VL comprises or consists of an amino acid sequence that is at least 90%, at least 95%, at least 97% identical, or 100% identical to SEQ ID NO: 55.

51. The method of claim 33, wherein the VH comprises or consists of an amino acid sequence that is at least 90%, at least 95%, at least 97% identical, or 100% identical to SEQ ID NO: 51 and the VL comprises or consists of an amino acid sequence that is at least 90%, at least 95%, at least 97% identical, or 100% identical to SEQ ID NO: 53.

52. The method of claim 33, wherein the VH comprises or consists of an amino acid sequence that is at least 90%, at least 95%, at least 97% identical, or 100% identical to SEQ ID NO: 51 and the VL comprises or consists of an amino acid sequence that is at least 90%, at least 95%, at least 97% identical, or 100% identical to SEQ ID NO: 54.

53. The method of claim 33, wherein the VH comprises or consists of an amino acid sequence that is at least 90%, at least 95%, at least 97% identical, or 100% identical to SEQ ID NO: 51 and the VL comprises or consists of an amino acid sequence that is at least 90%, at least 95%, at least 97% identical, or 100% identical to SEQ ID NO: 55.

54. The method of claim 33, wherein the VH comprises or consists of an amino acid sequence that is at least 90%, at least 95%, at least 97% identical, or 100% identical to SEQ ID NO: 52 and the VL comprises or consists of an amino acid sequence that is at least 90%, at least 95%, at least 97% identical, or 100% identical to SEQ ID NO: 53.

55. The method of claim 33, wherein the VH comprises or consists of an amino acid sequence that is at least 90%, at least 95%, at least 97% identical, or 100% identical to SEQ ID NO: 52 and the VL comprises or consists of an amino acid sequence that is at least 90%, at least 95%, at least 97% identical, or 100% identical to SEQ ID NO: 54.

56. The method of claim 33, wherein the VH comprises or consists of an amino acid sequence that is at least 90%, at least 95%, at least 97% identical, or 100% identical to SEQ ID NO: 52 and the VL comprises or consists of an amino acid sequence that is at least 90%, at least 95%, at least 97% identical, or 100% identical to SEQ ID NO: 55.

57. The method of any one of claims 33-56, wherein the anti-ceramide antibody or antigen-binding fragment thereof is a humanized 6B5 (h6B5) antibody.

58. The method of any one of claims 33-56, wherein the anti-ceramide antibody or antigen-binding fragment thereof is a h6B5 scFv.

59. The method of any one of claims 1-24, wherein the anti-ceramide antibody or antigen-binding fragment thereof comprises a variable heavy chain (V_{H}) and a variable light chain (V_{L}), wherein the V_{H} comprises an amino acid sequence of SEQ ID NO: 48, and wherein the V_{L} comprises an amino acid sequence of SEQ ID NO: 53.

60. The method of any one of claims 1-24, wherein the anti-ceramide antibody or antigen-binding fragment thereof comprises a variable heavy chain (V_{H}) and a variable light chain (V_{L}), wherein the V_{H} comprises an amino acid sequence of SEQ ID NO: 48, and wherein the V_{L} comprises an amino acid sequence of SEQ ID NO: 55.

61. The method of any one of claims 1-24, wherein the anti-ceramide antibody or antigen-binding fragment thereof comprises a variable heavy chain (V_{H}) and a variable light chain (V_{L}), wherein the V_{H} comprises an amino acid sequence of SEQ ID NO: 49, and wherein the V_{L} comprises an amino acid sequence of SEQ ID NO: 53.

62. The method of any one of claims 1-24, wherein the anti-ceramide antibody or antigen-binding fragment thereof comprises a variable heavy chain (V_{H}) and a variable light chain (V_{L}), wherein the V_{H} comprises an amino acid sequence of SEQ ID NO: 49, and wherein the V_{L} comprises an amino acid sequence of SEQ ID NO: 54.

63. The method of any one of claims 1-24, wherein the anti-ceramide antibody or antigen-binding fragment thereof comprises a variable heavy chain (V_{H}) and a variable light chain (V_{L}), wherein the V_{H} comprises an amino acid sequence of SEQ ID NO: 50, and wherein the V_{L} comprises an amino acid sequence of SEQ ID NO: 53.

64. The method of any one of claims 1-24, wherein the anti-ceramide antibody or antigen-binding fragment thereof comprises a variable heavy chain (V_{H}) and a variable light chain (V_{L}), wherein the V_{H} comprises an amino acid sequence of SEQ ID NO: 50, and wherein the V_{L} comprises an amino acid sequence of SEQ ID NO: 54.

65. The method of any one of claims 1-24, wherein the anti-ceramide antibody or antigen-binding fragment thereof comprises a variable heavy chain (V_{H}) and a variable light chain (V_{L}), wherein the V_{H} comprises an amino acid sequence of SEQ ID NO: 51, and wherein the V_{L} comprises an amino acid sequence of SEQ ID NO: 53.

66. The method of any one of claims 1-24, wherein the anti-ceramide antibody or antigen-binding fragment thereof comprises a variable heavy chain (V_{H}) and a variable light chain (V_{L}), wherein the V_{H} comprises an amino acid sequence of SEQ ID NO: 52, and wherein the V_{L} comprises an amino acid sequence of SEQ ID NO: 53.

67. The method of any one of claims 59-66, wherein the anti-ceramide antibody or antigen-binding fragment thereof is a humanized antibody.

68. The method of any one of claims 59-66, wherein the anti-ceramide antibody or antigen-binding fragment thereof is a humanized scFv.
